(19) United States
(12) Patent Application Publication
(10) Pub. No.: US 2016/0039994 A1
(43) Pub. Date: Feb. 11, 2016

(54) OPTICAL FILM, POLARIZING PLATE AND LIQUID CRYSTAL DISPLAY DEVICE

(71) Applicant: FUJIFILM Corporation, Tokyo (JP)

(72) Inventors: Naozumi SHIRAIWA, Kanagawa (JP); Hiroshi INADA, Kanagawa (JP); Aiko YOSHIDA, Kanagawa (JP); Yasukazu KUWAYAMA, Kanagawa (JP)

(73) Assignee: FUJIFILM CORPORATION, Tokyo (JP)

(21) Appl. No.: 14/884,314

(22) Filed: Oct. 15, 2015

Related U.S. Application Data
(63) Continuation of application No. PCT/JP2014/060786, filed on Apr. 16, 2014.

(30) Foreign Application Priority Data
Apr. 19, 2013 (JP) 2013-088451
Apr. 11, 2014 (JP) 2014-081511

(51) Int. Cl. C08K 5/3417; G02B 5/30; G02B 1/14; C07D 209/48; G02F 1/1335

(52) U.S. Cl. CPC C08K 5/3417; C07D 209/48; G02F 1/133528; G02B 1/14; G02B 5/305

(57) ABSTRACT

Provided is an optical film which is excellent in adhesiveness with a hard coat layer after a light resistance test, excellent in surface hardness, and excellent in the shape of the surface. The optical film contains cellulose acylate and a compound represented by the following Formula (I). In Formula (I), L represents a linking group of (n1+n2) valence of which the number of atoms linking A¹ to A² is less than or equal to 8; here, n1+n2 represents an integer of greater than or equal to 2, n1 represents an integer of greater than or equal to 1, and n2 represents an integer of greater than or equal to 0; and A¹ represents a group represented by Formula (II) and A² represents a group represented by Formula (III).

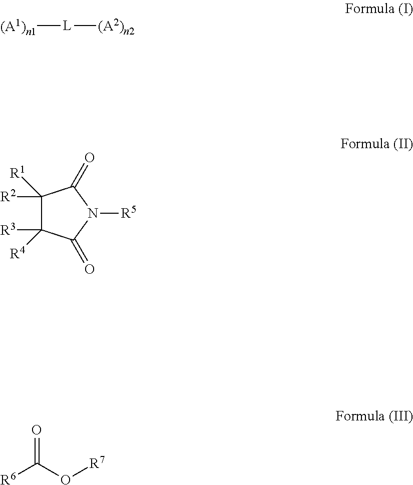

### CROSS-REFERENCE TO RELATED APPLICATIONS

This application is a Continuation of PCT International Application No. PCT/JP2014/060786 filed on Apr. 16, 2014, which claims priority under 35 U.S.C §119(a) to Japanese Patent Application No. 2013-088451 filed on Apr. 19, 2013 and Japanese Patent Application No. 2014-081511 filed on Apr. 11, 2014. Each of the above application(s) is hereby expressly incorporated by reference, in its entirety, into the present application.

### BACKGROUND OF THE INVENTION

1. Field of the Invention

The present invention relates to an optical film, a polarizing plate, and a liquid crystal display device. Particularly, an optical film which is useful as a viewer-side polarizing plate protective film, a polarizing plate using the same, and a liquid crystal display device having the above-described polarizing plate.

2. Description of the Related Art

In recent years, an increase in size and a reduction in thickness of a liquid crystal display device have progressed mainly for an application of TVs, and accordingly, it is also necessary to reduce the thickness of an optical film which is a constituent member. In the related art, appropriate hardness and favorable cutting properties of an optical film have been considered to be important as well in view of processability. In an optical film of which the thickness is reduced, improvement in the hardness and the cutting properties is further expected.

Particularly, in a case where the thickness of an optical film which is provided on a viewer-side of a display device such as a liquid crystal display device is reduced, the optical film is easily damaged if the hardness thereof is low. Therefore, in many cases, a hard coat layer is generally formed thereon in order to impart hardness to the optical film. Even in the optical film having such a hard coat layer, it is important to improve the hardness of a base film in order to improve the hardness of the optical film.

In contrast, technology of adding a compound disclosed in JP2012-123292A and JP2009-15045A to an optical film as an additive is known (JP2012-123292A and JP2009-15045A).

### SUMMARY OF THE INVENTION

JP2012-123292A discloses an additive for exhibiting high retardation and improving brittleness and durability. In addition, JP2009-15045A discloses an additive for improving surface processability, high contrast, and high hygrothermal durability. However, as a result of reviewing the additives disclosed in JP2012-123292A and JP2009-15045A by the present inventors, it was found that there is a problem in adhesiveness with hard coat layers of these optical films after a light resistance test.

In addition, in a liquid crystal display device of which the thickness is reduced as described above, appropriate surface hardness in a film is expected and a uniform film which is excellent in transparency is also expected in order to realize favorable display performance. For this reason, it is necessary to suppress a haze of the film.

The present invention has been made in order to solve the problems, and an object of the present invention is to provide an optical film which has appropriate surface hardness, is excellent in adhesiveness with a hard coat layer after a light resistance test, and has low haze.

The present inventors have conducted extensive studies for the above-described purposes, and as a result, they have found that the additives disclosed in JP2012-123292A and JP2009-15045A have a configuration in which light of an ultraviolet region included in natural light is easily absorbed, and therefore, there is a problem in adhesiveness of a hard coat layer after a light resistance test. Thus, it was found that the present invention is excellent in adhesiveness with a hard coat layer after a light resistance test using a compound represented by Formula (I) to be described below. Furthermore, it was found that it is possible to provide an optical film which is excellent in surface hardness and in suppressing a haze of the film when the compound represented by Formula (I) to be described below is formulated in. In this manner, the present inventors have completed the present invention. The present invention is undeterred by any theory. However, it is considered that the hardness of an optical film is improved since the compound represented by Formula (I) interacts with a polymer chain or a local site, such as an ester bond or a hydroxyl group, of cellulose acylate and a interspace between polymer chains of the cellulose acylate existing in the film, that is, the free volume is reduced.

Specifically, the above-described problems have been solved by the following means <1> or preferably by the following means <2> to <18>.

<1> An optical film including:
cellulose acylate; and
a compound represented by the following Formula (I).

(A¹)ₙ₁-L-(A²)ₙ₂ Formula (I)

(In Formula (I), L represents a linking group of (n1+n2) valence of which the number of atoms linking A¹ to A² is less than or equal to 8. Here, n1+n2 represents an integer of greater than or equal to 2, n1 represents an integer of greater than or equal to 1, and n2 represents an integer of greater than or equal to 0. A¹ represents a group represented by Formula (II) and A² represents a group represented by Formula (III).)

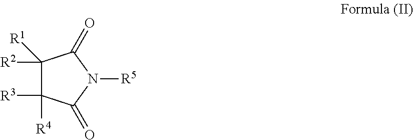

(In Formula (II), R¹ to R⁴ each independently represent a hydrogen atom, a substituted or unsubstituted alkyl group, a substituted or unsubstituted acyl group, a substituted or unsubstituted alkoxycarbonyl group, a substituted or unsubstituted alkoxy group, a substituted or unsubstituted aryloxy group, a substituted or unsubstituted alkylthio group, a substituted or unsubstituted arylthio group, a substituted or unsubstituted carbamoyl group, a carboxyl group, a cyano group, or a hydroxyl group, and R¹ to R⁴ may form a non-aromatic ring by being bonded to each other. R⁵ represents a hydrogen atom or a substituted or unsubstituted alkyl group. Here, one of R¹ to R⁵ represents a bonding site with L in Formula (I).)

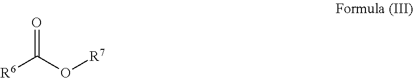

(In Formula (III), R⁶ and R⁷ each independently represent a substituted or unsubstituted phenyl group or a substituted or unsubstituted alkyl group. Here, at least one of R⁶ and R⁷ represents a bonding site with L in Formula (I).)

<2> The optical film according to <1>,
in which, in Formula (I), L contains at least one of a tertiary carbon atom and a quaternary carbon atom.

<3> The optical film according to <1> or <2>,
in which, in Formula (I), L represents a linking group of (n1+n2) valence of which the number of atoms linking A¹ to A² is less than or equal to 5.

<4> The optical film according to any one of <1> to <3>,
in which, the molecular weight of the compound represented by Formula (I) is 300 to 1000.

<5> The optical film according to any one of <1> to <4>,
in which, in Formula (II), the bonding site with L is R⁵.

<6> The optical film according to any one of <1> to <5>,
in which, in Formula (I), n1 represents an integer of 2 to 5 and n2 represents 0.

<7> The optical film according to <6>,
in which, in Formula (I), n1 represents an integer of 2 or 3.

<8> The optical film according to any one of <1> to <7>,
in which, in Formula (II), R¹ to R⁴ bond to each other to form a 5- or 6-membered aliphatic ring.

<9> The optical film according to any one of <1> to <5> and <8>,
in which, in Formula (I), n1 represents 1 and n2 represents an integer of 1 to 5.

<10> The optical film according to any one of <1> to <5>, <8>, and <9>,
in which, in Formula (III), the bonding site with L is R⁷.

<11> The optical film according to any one of <1> to <10>,
in which, in Formula (II), R¹ to R⁴ each independently represent a hydrogen atom, an alkyl group, an acyl group, an alkoxycarbonyl group, a carbamoyl group, an alkoxy group, an aryloxy group, an alkylthio group, an arylthio group, a cyano group, or a hydroxyl group.

<12> The optical film according to any one of <1> to <5> and <8> to <11>,
in which, in Formula (III), R⁶ represents a substituted or unsubstituted phenyl group or a substituted or unsubstituted alkyl group.

<13> The optical film according to any one of <1> to <12>,
in which the content of the compound represented by Formula (I) is 3 parts by mass to 60 parts by mass with respect to 100 parts by mass of cellulose acylate.

<14> The optical film according to any one of <1> to <13>,
in which the optical film is used for a protective film of a viewer-side polarizing plate.

<15> The optical film according to any one of <1> to <14>, further including:
a hard coat layer.

<16> A polarizing plate including:
the optical film according to any one of <1> to <15>; and
a polarizer.

<17> A liquid crystal display device including:
the polarizing plate according to <16>.

<18> A compound which is represented by the following Formula (I-3) or (I-5).

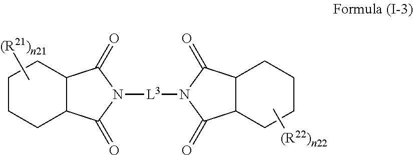

(In Formula (I-3), L³ represents a divalent linking group which contains at least one of a tertiary carbon atom and a quaternary carbon atom and of which the number of atoms linking nitrogen atoms together is less than or equal to 8. R²¹ and R²² represent a methyl group. n21 and n22 each represent an integer of 0 or 1.)

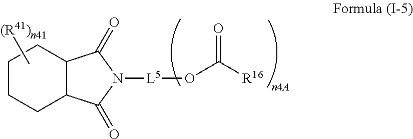

(In Formula (I-5), L⁵ represents a linking group which is selected from the following linking group A, R⁴¹ represents a methyl group, and n41 represents an integer of 0 or 1. R¹⁶ represents a substituent which is selected from the following substituent group B and a plurality of the substituents may be the same as or different from each other. n4A represents an integer of 1 to 3.)

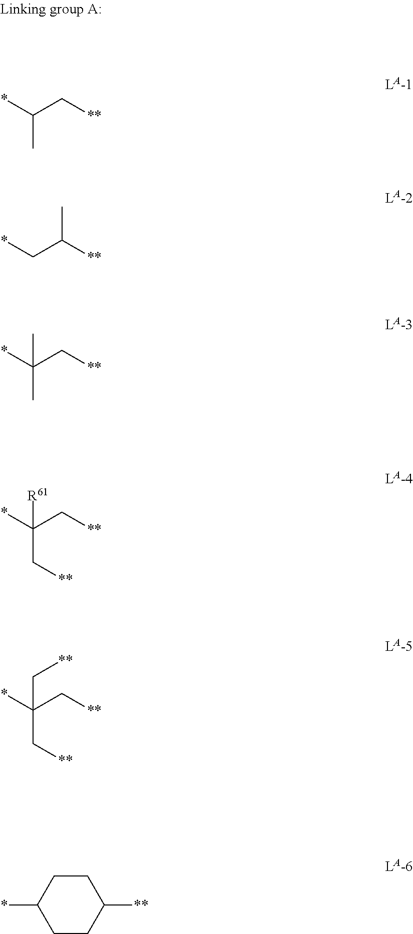

(* represents a bonding site with a nitrogen atom and ** represents a bonding site with an oxygen atom. R⁵¹ represents a hydrogen atom, a methyl group, or an ethyl group.)

Substituent group B:

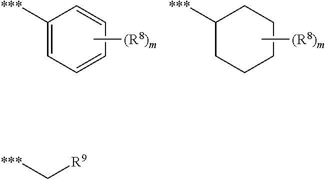

(*** represents a bonding site with a carbonyl group, R⁸ represents an alkyl group, an alkoxycarbonyl group, an acyl group, or an alkoxy group, and R⁹ represents a carbonyl group, an alkoxycarbonyl group, a carbonyloxy group, a cyano group, or a hydroxyl group. m represents an integer of 0 to 3.)

According to the present invention, it is possible to provide an optical film which is excellent in adhesiveness with a hard coat layer after a light resistance test, excellent in surface hardness, and has a low haze.

### BRIEF DESCRIPTION OF THE DRAWING

FIG. 1 is a schematic view showing a positional relationship between a polarizing plate and a liquid crystal display device of the present invention.

### DESCRIPTION OF THE PREFERRED EMBODIMENTS

Hereafter, the content of the present invention will be described in detail. In the present specification, “to” is used with a meaning which includes numerical values written before and after “to” as a lower limit value and an upper limit value. In the present specification, the “group” such as an alkyl group may have or may not have a substituent unless otherwise specified. Furthermore, in a case of a group in which the number of carbon atoms is limited, the above-described number of carbon atoms means the number of carbon atoms including the number of carbon atoms contained in a substituent.

<Optical Film>

An optical film of the present invention includes cellulose acylate and a compound represented by the following Formula (I). With use of such a compound, it is possible to obtain an optical film which is excellent in adhesiveness with a hard coat layer after a light resistance test, excellent in surface hardness, and excellent in the shape of the surface.

(A¹)ₙ₁-L-(A²)ₙ₂ Formula (I)

(In Formula (I), L represents a linking group of (n1+n2) valence of which the number of atoms linking A¹ to A² is less than or equal to 8. Here, n1+n2 represents an integer of greater than or equal to 2, n1 represents an integer of greater than or equal to 1, and n2 represents an integer of greater than or equal to 0. A¹ represents a group represented by Formula (II) and A² represents a group represented by Formula (III).)

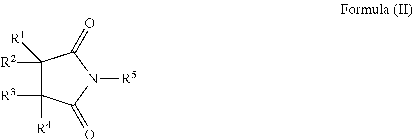

(In Formula (II), R¹ to R⁴ each independently represent a hydrogen atom, a substituted or unsubstituted alkyl group, a substituted or unsubstituted acyl group, a substituted or unsubstituted alkoxycarbonyl group, a substituted or unsubstituted alkoxy group, a substituted or unsubstituted aryloxy group, a substituted or unsubstituted alkylthio group, a substituted or unsubstituted arylthio group, a substituted or unsubstituted carbamoyl group, a carboxyl group, a cyano group, or a hydroxyl group, and R¹ to R⁴ may form a non-aromatic ring by being bonded to each other. R⁵ represents a hydrogen atom or a substituted or unsubstituted alkyl group. Here, one of R¹ to R⁵ represents a bonding site with L in Formula (I).)

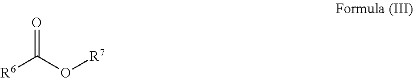

(In Formula (III), R⁶ and R⁷ each independently represent a substituted or unsubstituted phenyl group or a substituted or unsubstituted alkyl group. Here, at least one of R⁶ and R⁷ represents a bonding site with L in Formula (I).)

<<Formula (I)>>

In Formula (I), L represents a linking group of (n1+n2) valence of which the number of atoms linking A¹ to A² is less than or equal to 8. The size of the molecular structure becomes relatively small by making the number of atoms linking A¹ to A² be less than or equal to 8. Therefore, it is possible to reduce the free volume existing in cellulose acylate through interaction between the compound and with a polymer chain or a local site, such as an ester bond or a hydroxyl group, of cellulose acylate, in the cellulose acylate, and as a result, it is possible to improve the hardness of the film. As L, a linking group of (n1+n2) valence of which the number of atoms linking A¹ to A² is less than or equal to 5 is preferable and a linking group of (n1+n2) valence of which the number of atoms linking A¹ to A² is 2 to 5 is more preferable.

Here, the number of atoms linking A¹ to A² refers to the shortest number of atoms linking A¹ to A². In a case where there are a plurality of Ls in the same molecule, the number of atoms linking A¹ to A² is obtained by counting the number of atoms in the longest linking group among them. For example, the number of atoms of the following compound becomes 2.

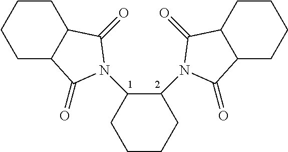 [SMILES: O=C1C2CCCCC2C(=O)N1C1CCCCC1N1C(=O)C2CCCCC2C1=O]

L preferably contains at least one of a tertiary carbon atom and a quaternary carbon atom. With such a configuration, the above-described interaction between the cellulose acylate and the compound represented by Formula (I) is heightened. Therefore, the hardness of the film tends to be more improved.

L is preferably a group including at least one kind of a straight-chain, branched, or cyclic aliphatic group and aromatic group and more preferably a group containing a branched or cyclic aliphatic group. L may be at least only one kind of a straight-chain, branched, or cyclic aliphatic group and aromatic group, or these groups are preferably combined with an oxygen atom, a nitrogen atom, a carbonyl group, a straight-chain or a branched alkylene group, and a straight-chain or branched arylene group.

The aliphatic group contained in L may be any of a saturated aliphatic group and an unsaturated aliphatic group, and is preferably a saturated aliphatic group.

Examples of the branched or cyclic aliphatic group contained in L include 1-methyl ethylene group, 1,3-cyclohexylene group, and 1,2-cyclohexylene group.

Specifically, the linking group represented by L is preferably a linking group exemplified below. * represents a position which is bonded to A¹ or A². R represents a hydrogen atom, a methyl group, or an ethyl group.

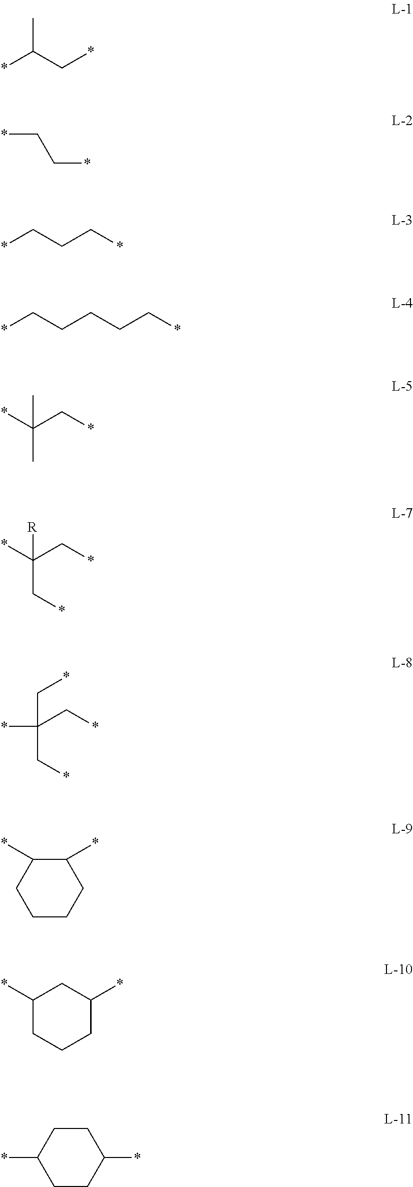

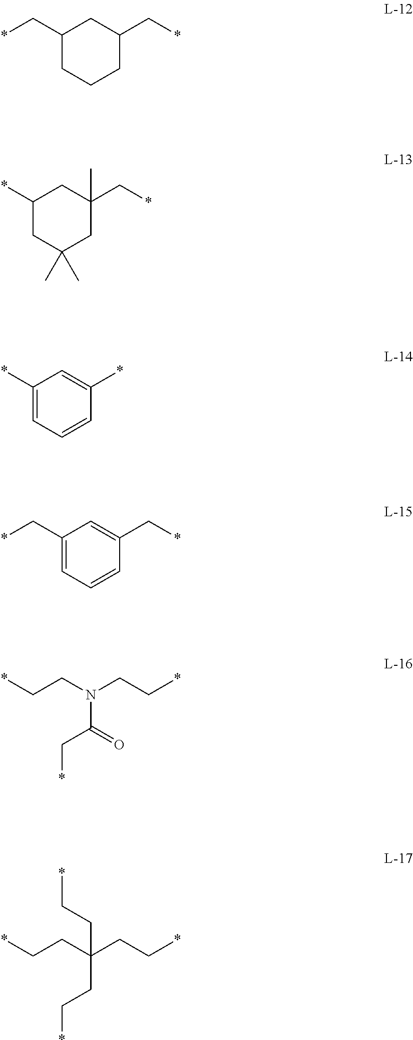 [SMILES: *CC1CCCC(C*)C1 ; *CC1(C)CC(*)CC(C)(C)C1 ; *c1cccc(*)c1 ; *Cc1cccc(C*)c1 ; *CCN(CC*)C(=O)C* ; *CCC(CC*)(CC*)CC*]

Among these linking groups represented by L, linking groups exemplified by L-1, L-2, L-5, and L-7 to L-15 are more preferable. Among the above, linking groups exemplified by L-1, L-2, L-5, L-8 to L-10, and L-12 to L-15 are still more preferable.

A linking group represented by L may further have a substituent, and examples of the substituent include the following substituent group T. The linking group represented by L preferably has no substituent.

Substituent group T:

Alkyl groups (preferably having 1 to 10 carbon atoms, more preferably having 1 to 8 carbon atoms, and particularly preferably having 1 to 6 carbon atoms, and examples thereof include a methyl group, an ethyl group, an isopropyl group, a tert-butyl group, an n-octyl group, an n-decyl group, an n-hexadecyl group, a cyclopropyl group, cyclopentyl group, and a cyclohexyl group); alkenyl groups (preferably having 2 to 10 carbon atoms, more preferably having 2 to 8 carbon atoms, and particularly preferably having 2 to 6 carbon atoms, and examples thereof include a vinyl group, an allyl group, a 2-butenyl group, and a 3-pentenyl group); alkynyl groups (preferably having 2 to 10 carbon atoms, more preferably having 2 to 8 carbon atoms, and particularly preferably having 2 to 6 carbon atoms, and examples thereof include a propargyl group and a 3-pentynyl group); aryl groups (preferably having 6 to 20 carbon atoms, more preferably having 6 to 15 carbon atoms, and particularly preferably having 6 to 12 carbon atoms, and examples thereof include a phenyl group, a biphenyl group, and a naphthyl group); amino groups (preferably having 0 to 15 carbon atoms, more preferably, having 0 to 10 carbon atoms, and particularly preferably having 0 to 6 carbon atoms, and examples thereof include an amino group, a methyl amino group, a dimethyl amino group, a diethyl amino group, and a dibenzyl amino group); alkoxy groups (preferably having 1 to 10 carbon atoms, more preferably having 1 to 8 carbon atoms, and particularly preferably having 1 to 6 carbon atoms, and examples thereof include a methoxy group, an ethoxy group, and a butoxy group); aryloxy groups (preferably having 6 to 20 carbon atoms, more preferably having 6 to 15 carbon atoms, and particularly preferably having 6 to 12 carbon atoms, and examples thereof include a phenyloxy group, and a 2-naphthyloxy group); acyl groups (preferably having 1 to 15 carbon atoms, more preferably having 1 to 10 carbon atoms, and particularly preferably having 1 to 8 carbon atoms, and examples thereof include an acetyl group, a benzoyl group, a formyl group, and a pivaloyl group); alkoxycarbonyl groups (preferably having 2 to 15 carbon atoms, more preferably having 2 to 10 carbon atoms, and particularly preferably having 2 to 8 carbon atoms, and examples thereof include a methoxycarbonyl group and an ethoxycarbonyl group); aryloxycarbonyl groups (preferably having 7 to 20 carbon atoms, more preferably having 7 to 16 carbon atoms, and particularly preferably having 7 to 10 carbon atoms, and examples thereof include a phenyloxycarbonyl group); acyloxy groups (preferably having 2 to 15 carbon atoms, more preferably having 2 to 10 carbon atoms, and particularly preferably having 2 to 8 carbon atoms, and examples thereof include an acetoxy group and a benzoyloxy group); acylamino groups (preferably having 2 to 15 carbon atoms, more preferably having 2 to 10 carbon atoms, and particularly preferably having 2 to 8 carbon atoms, and examples thereof include an acetylamino group and a benzoylamino group); alkoxycarbonylamino groups (preferably having 2 to 15 carbon atoms, more preferably having 2 to 10 carbon atoms, and particularly preferably having 2 to 8 carbon atoms, and examples thereof include a methoxycarbonylamino group); aryloxycarbonylamino groups (preferably having 7 to 15 carbon atoms, more preferably having 7 to 13 carbon atoms, and particularly preferably having 7 to 10 carbon atoms, and examples thereof include a phenyloxycarbonylamino group); sulfonylamino groups (preferably having 1 to 15 carbon atoms, more preferably having 1 to 10 carbon atoms, and particularly preferably having 1 to 8 carbon atoms, and examples thereof include a methanesulfonylamino group and a benzenesulfonylamino group); sulfamoyl groups (preferably having 0 to 15 carbon atoms, more preferably having 0 to 10 carbon atoms, and particularly preferably having 0 to 8 carbon atoms, and examples thereof include a sulfamoyl group, a methyl sulfamoyl group, a dimethylsulfamoyl group, and a phenylsulfamoyl group); carbamoyl groups (preferably having 1 to 15 carbon atoms, more preferably having 1 to 10 carbon atoms, and particularly preferably having 1 to 8 carbon atoms, and examples thereof include a carbamoyl group, a methylcarbamoyl group, a diethylcarbamoyl group, and a phenylcarbamoyl group); alkylthio groups (preferably having 1 to 15 carbon atoms, more preferably having 1 to 10 carbon atoms, and particularly preferably having 1 to 8 carbon atoms, and examples thereof include a methylthio group and an ethylthio group); arylthio groups (preferably having 6 to 20 carbon atoms, more preferably having 6 to 15 carbon atoms, and particularly preferably having 6 to 12 carbon atoms, and examples thereof include a phenylthio group); sulfonyl groups (preferably having 1 to 20 carbon atoms, more preferably having 1 to 16 carbon atoms, and particularly preferably having 1 to 12 carbon atoms, and examples thereof include a mesyl group and a tosyl group); sulfinyl groups (preferably having 1 to 20 carbon atoms, more preferably having 1 to 16 carbon atoms, and particularly preferably having 1 to 12 carbon atoms, and examples thereof include a methane sulfinyl group and a benzene sulfinyl group); ureido groups (preferably having 1 to 20 carbon atoms, more preferably having 1 to 16 carbon atoms, and particularly preferably having 1 to 12 carbon atoms, and examples thereof include a ureido group, a methylureido group, and a phenyl ureido group); phosphoric acid amide groups (preferably having 1 to 20 carbon atoms, more preferably having 1 to 16 carbon atoms, and particularly preferably having 1 to 12 carbon atoms, and examples thereof include diethyl phosphoric acid amide and phenyl phosphoric acid amide); hydroxy groups; mercapto groups; halogen atoms (for example, a fluorine atom, a chlorine atom, a bromine atom, and an iodine atom); cyano groups; sulfo groups; carboxyl groups; nitro groups; hydroxamic acid groups; sulfide groups; hydrazino groups; imino groups; heterocyclic groups (preferably having 1 to 20 carbon atoms and more preferably having 1 to 12 carbon atoms, and examples of hetero atoms include a nitrogen atom, an oxygen atom, and a sulfur atom, and specific examples thereof include an imidazolyl group, a pyridyl group, a quinolyl group, a furyl group, a piperidyl group, a morpholino group, a benzoxazolyl group, a benzimidazolyl group, and a benzothiazolyl group); and silyl groups (preferably having 3 to 20 carbon atoms, more preferably having 3 to 15 carbon atoms, and particularly preferably having 3 to 10 carbon atoms, and examples thereof include a trimethylsilyl group and a triphenylsilyl group).

These substituents may further be substituted. In addition, in a case where there are two or more substituents, the substituents may be the same as or different from each other. In addition, the substituents may form a ring by being linked to each other if possible.

Among the above, as a substituent processed by the linking group represented by L, an alkyl group, an alkenyl group, an aryl group, an alkoxy group, an aryloxy group, an acyl group, an alkoxycarbonyl group, an acyloxy group, an acylamino group, an alkoxycarbonylamino group, a carbamoyl group, an alkylthio group, a ureido group, a hydroxyl group, a cyano group, a carboxyl group, or a silyl group is preferable.

n1+n2 represents an integer of greater than or equal to 2, n1 represents an integer of greater than or equal to 1, and n2 represents an integer of greater than or equal to 0. n1+n2 is preferably an integer of 2 to 5 and more preferably an integer of 2 to 4. n1 is preferably an integer of 1 to 5 and more preferably an integer of 1 to 3. n2 is preferably an integer of 0 to 4 and more preferably an integer of 0 to 3.

As the compound represented by Formula (I), in a case where n1 represents an integer of 2 to 5, n2 preferably represents 0 to 2 and more preferably represents 0. In a case where n1 represents an integer of 2 or 3, n2 more preferably represents 0. It is particularly preferable that n1 represents 2 and n2 represents 0.

In addition, as the compound represented by Formula (I), in a case where n1 represents 1, n2 preferably represents an integer of 1 to 5 and n2 more preferably represents an integer of 2 or 3.

<Formula (II)>>

A¹ represents a group represented by Formula (II).

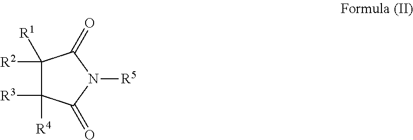

(In Formula (II), R¹ to R⁴ each independently represent a hydrogen atom, a substituted or unsubstituted alkyl group, a substituted or unsubstituted acyl group, a substituted or unsubstituted alkoxycarbonyl group, a substituted or unsubstituted alkoxy group, a substituted or unsubstituted aryloxy group, a substituted or unsubstituted alkylthio group, a substituted or unsubstituted arylthio group, a substituted or unsubstituted carbamoyl group, a carboxyl group, a cyano group, or a hydroxyl group, and R¹ to R⁴ may form a non-aromatic ring by being bonded to each other. R⁵ represents a hydrogen atom or a substituted or unsubstituted alkyl group. Here, one of R¹ to R⁵ represents a bonding site with L.)

R¹ to R⁴ each independently represent a hydrogen atom, a substituted or unsubstituted alkyl group, a substituted or unsubstituted acyl group, a substituted or unsubstituted alkoxycarbonyl group, a substituted or unsubstituted alkoxy group, a substituted or unsubstituted aryloxy group, a substituted or unsubstituted alkylthio group, a substituted or unsubstituted arylthio group, a substituted or unsubstituted carbamoyl group, a carboxyl group, a cyano group, or a hydroxyl group. Since R¹ to R⁴ do not have a double bond directly bonded to an imide structure, it is possible to shortwave absorption of light and to suppress the absorption of light in an ultraviolet region which is contained in natural light. As a result, it is possible to suppress deterioration of cellulose acylate due to light irradiation when the compound represented by Formula (I) is made to be contained in the optical film. Therefore, it is possible to improve light-resistant adhesiveness.

As the substituted or unsubstituted alkyl group, an alkyl group having 1 to 15 carbon atoms is preferable, an alkyl group having 1 to 10 carbon atoms is more preferable, and an alkyl group having 1 to 6 carbon atoms is still more preferable.

As the substituted or unsubstituted acyl group, an acyl group having 1 to 15 carbon atoms is preferable, an acyl group having 1 to 10 carbon atoms is more preferable, and an acyl group having 1 to 6 carbon atoms is still more preferable.

As the substituted or unsubstituted alkoxycarbonyl group, an alkoxycarbonyl group having 1 to 15 carbon atoms is preferable, an alkoxycarbonyl group having 1 to 10 carbon atoms is more preferable, and an alkoxycarbonyl group having 1 to 6 carbon atoms is still more preferable.

As the substituted or unsubstituted alkoxy group, an alkoxy group having 1 to 15 carbon atoms is preferable, an alkoxy group having 1 to 10 carbon atoms is more preferable, and an alkoxy group having 1 to 6 carbon atoms is still more preferable.

As the substituted or unsubstituted aryloxy group, an aryloxy group having 6 to 20 carbon atoms is preferable, an aryloxy group having 6 to 15 carbon atoms is more preferable, and an aryloxy group having 6 to 12 carbon atoms is still more preferable.

As the substituted or unsubstituted alkylthio group, an alkylthio group having 1 to 15 carbon atoms is preferable, an alkylthio group having 1 to 10 carbon atoms is more preferable, and an alkylthio group having 1 to 6 carbon atoms is still more preferable.

As the substituted or unsubstituted arylthio group, an arylthio group having 6 to 20 carbon atoms is preferable, an arylthio group having 6 to 15 carbon atoms is more preferable, and an arylthio group having 6 to 12 carbon atoms is still more preferable.

As the substituted or unsubstituted carbamoyl group, an alkyl group having 1 to 15 carbon atoms is preferable, an alkyl group having 1 to 10 carbon atoms is more preferable, and an alkyl group having 1 to 6 carbon atoms is still more preferable.

These groups may be straight-chain, branched, or cyclic groups. Examples of a substituent which may be included in an alkyl group, an acyl group, an alkoxycarbonyl group, an alkoxy group, an aryloxy group, an alkylthio group, a carbamoyl group, and an arylthio group include the substituent group T. Among these, an alkyl group, an alkenyl group, an aryl group, an alkoxy group, an aryloxy group, an acyl group, an alkoxycarbonyl group, an acyloxy group, an acylamino group, an alkoxycarbonylamino group, a carbamoyl group, an alkylthio group, a ureido group, a hydroxy group, a cyano group, a carboxyl group, or a silyl group is preferable.

R¹ or R² and R³ or R⁴ may be bonded to each other to form a non-aromatic ring. The non-aromatic ring is called a ring other than an aromatic ring, and may be a monocyclic ring or may form a polycyclic ring greater than or equal to bicycle. As the non-aromatic ring, a 5- or 6-membered monocyclic or polycyclic aliphatic ring is preferable, a 5- or 6-membered aliphatic ring is more preferable, and a cyclohexane ring or a cyclohexene ring is still more preferable.

R⁵ represents a hydrogen atom or a substituted or unsubstituted alkyl group. As the substituted or unsubstituted alkyl group, an alkyl group having 1 to 20 carbon atoms is preferable, an alkyl group having 1 to 10 carbon atoms is more preferable, and an alkyl group having 1 to 6 carbon atoms is still more preferable.

One of R¹ to R⁵ represents a bonding site with L, and it is preferable that R⁵ is bonded to L (R⁵ is a bonding site with L).

Formula (II) is preferably represented by the following Formula (II-1).

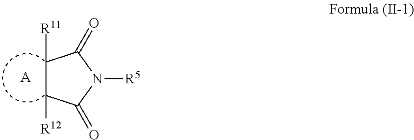

(In Formula (II-1), a ring A represents a non-aromatic ring, and R¹¹ and R¹² each independently represent a hydrogen atom, a substituted or unsubstituted alkyl group, a substituted or unsubstituted alkoxy group, a substituted or unsubstituted aryloxy group, a substituted or unsubstituted alkylthio group, or a substituted or unsubstituted arylthio group. R⁵ represents a hydrogen atom or a substituted or unsubstituted alkyl group. Here, one of R¹¹, R¹², and R⁵ represents a bonding site with L in Formula (I).)

The ring A represents a non-aromatic ring, and may be a monocyclic ring, may form a polycyclic ring greater than or equal to a bicycle, or may be a ring through an oxygen atom. In addition, the structure represented by Formula (II-1) may be linked to a cyclic structure which is the same as the ring A. As the non-aromatic ring, a 5- or 6-membered monocyclic or polycyclic aliphatic ring is preferable, a 5- or 6-membered aliphatic ring is more preferable, and a cyclohexane ring or a cyclohexene ring is still more preferable.

R¹¹ and R¹² each independently represent a hydrogen atom, a substituted or unsubstituted alkyl group, a substituted or unsubstituted alkoxy group, a substituted or unsubstituted aryloxy group, a substituted or unsubstituted alkylthio group, or a substituted or unsubstituted arylthio group. As the substituted or unsubstituted alkyl group, an alkyl group having 1 to 15 carbon atoms is preferable, an alkyl group having 1 to 10 carbon atoms is more preferable, an alkyl group having 1 to 6 carbon atoms is still more preferable, and an alkyl group having 1 to 3 carbon atoms is particularly preferable.

As the substituted or unsubstituted alkoxy group, an alkoxy group having 1 to 15 carbon atoms is preferable, an alkoxy group having 1 to 10 carbon atoms is more preferable, an alkoxy group having 1 to 6 carbon atoms is still more preferable, and an alkoxy group having 1 to 3 carbon atoms is particularly preferable.

As the substituted or unsubstituted aryloxy group, an aryloxy group having 6 to 20 carbon atoms is preferable, an aryloxy group having 6 to 15 carbon atoms is more preferable, an aryloxy group having 6 to 12 carbon atoms is still more preferable, and an aryloxy group having 6 to 10 carbon atoms is particularly preferable.

As the substituted or unsubstituted alkylthio group, an alkylthio group having 1 to 15 carbon atoms is preferable, an alkylthio group having 1 to 10 carbon atoms is more preferable, an alkylthio group having 1 to 6 carbon atoms is still more preferable, and an alkylthio group having 1 to 3 carbon atoms is particularly preferable.

As the substituted or unsubstituted arylthio group, an arylthio group having 6 to 20 carbon atoms is preferable, an arylthio group having 6 to 15 carbon atoms is more preferable, an arylthio group having 6 to 12 carbon atoms is still more preferable, and an arylthio group having 6 to 10 carbon atoms is particularly preferable.

Examples of a substituent which may be included in an alkyl group include the substituent group T, and preferably include an alkyl group, an alkenyl group, an aryl group, an alkoxy group, an aryloxy group, an acyl group, an alkoxycarbonyl group, an acyloxy group, an acylamino group, an alkoxycarbonylamino group, a carbamoyl group, an alkylthio group, a ureido group, a hydroxyl group, a cyano group, a carboxyl group, or a silyl group.

R⁵ in Formula (II-1) is synonymous with R⁵ in Formula (II), and a preferred range of R⁵ in Formula (II-1) is the same as that of R⁵ in Formula (II).

The bonding site with L in Formula (II-1) is preferably R⁵.

Specifically, the group represented by Formula (II) is preferably a group exemplified below. * represents a position which is bonded to L, ** represents a position which is bonded to a substituent R^{a} or R^{b} (R^{a} represents an alkyl group and R^{b} represents an alkyl group, a cyclic alkyl group, or an aryl group). The group represented by Formula (II) may be mixed with a geometric isomer or an enantiomer which is generated by the state of being bonded to L. Here, the substitution positions of substituents shown in A¹-27, A¹-32, and A¹-33 are not particularly determined, and may be substituted at any position on a 6-membered structure.

As the alkyl group represented by R^{a} or R^{b}, an alkyl group having 1 to 10 carbon atoms is preferable, an alkyl group having 1 to 5 carbon atoms is more preferable, and an alkyl group having 1 to 3 carbon atoms is still more preferable.

As the cyclic alkyl group represented by R^{b}, a cyclic alkyl group having 6 to 12 carbon atoms is preferable, a cyclic alkyl group having 6 to 8 carbon atoms is more preferable, and a cyclic alkyl group having 6 to 8 carbon atoms is still more preferable.

As the aryl group represented by R^{b}, an aryl group having 6 to 12 carbon atoms is preferable, an aryl group having 6 to 10 carbon atoms is more preferable, and an aryl group having 6 to 8 is still more preferable.

These groups may be straight-chain, branched, or cyclic groups. Examples of a substituent which may be included in an alkyl group, a cyclic alkyl group, and an aryl group include the substituent group T. Among these, an alkyl group, an alkenyl group, an aryl group, an alkoxy group, an aryloxy group, an acyl group, an alkoxycarbonyl group, an acyloxy group, an acylamino group, an alkoxycarbonylamino group, a carbamoyl group, an alkylthio group, a ureido group, a hydroxy group, a cyano group, a carboxyl group, or a silyl group is preferable.

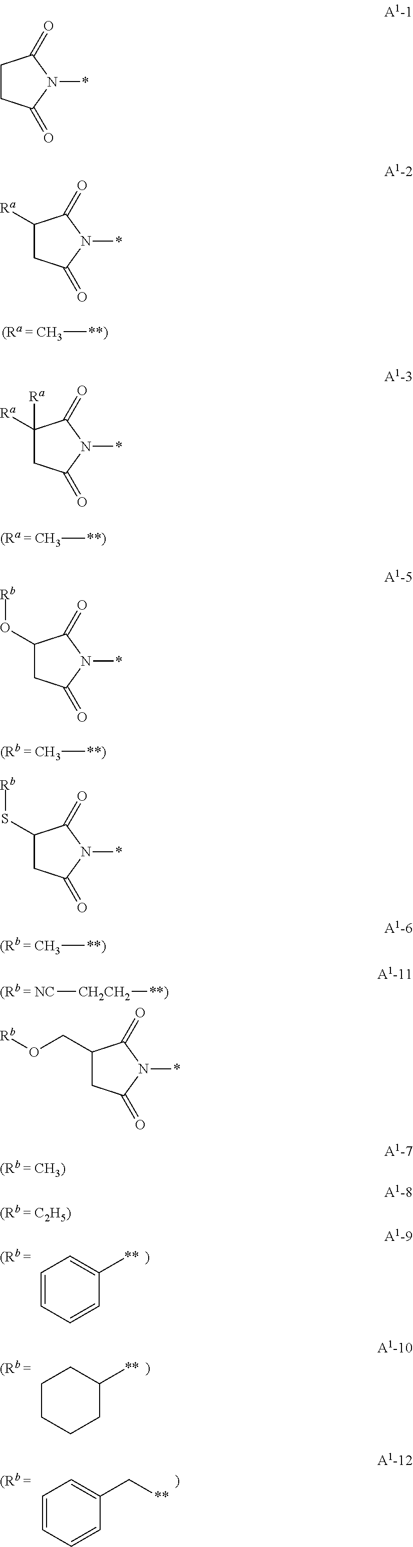

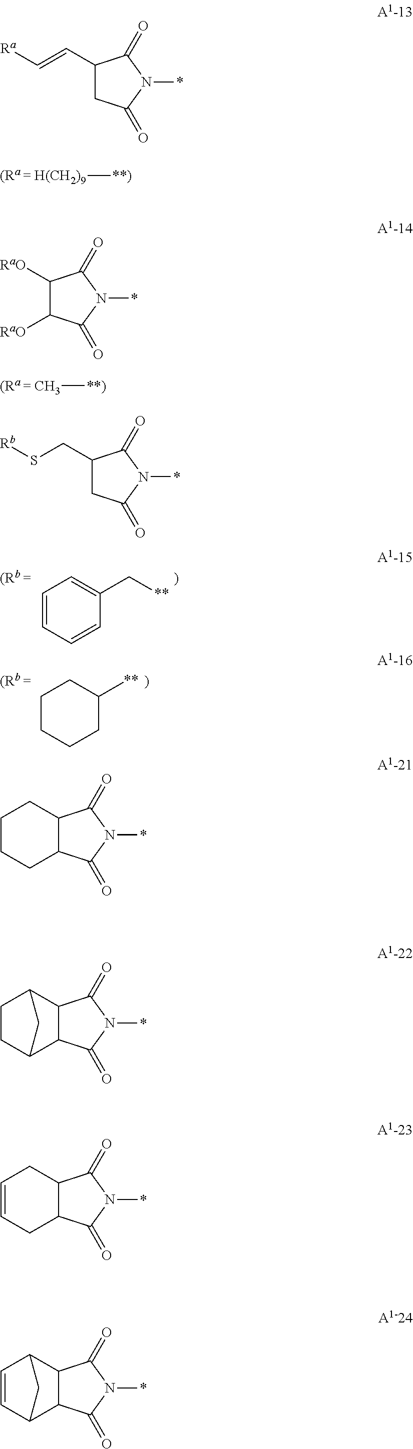

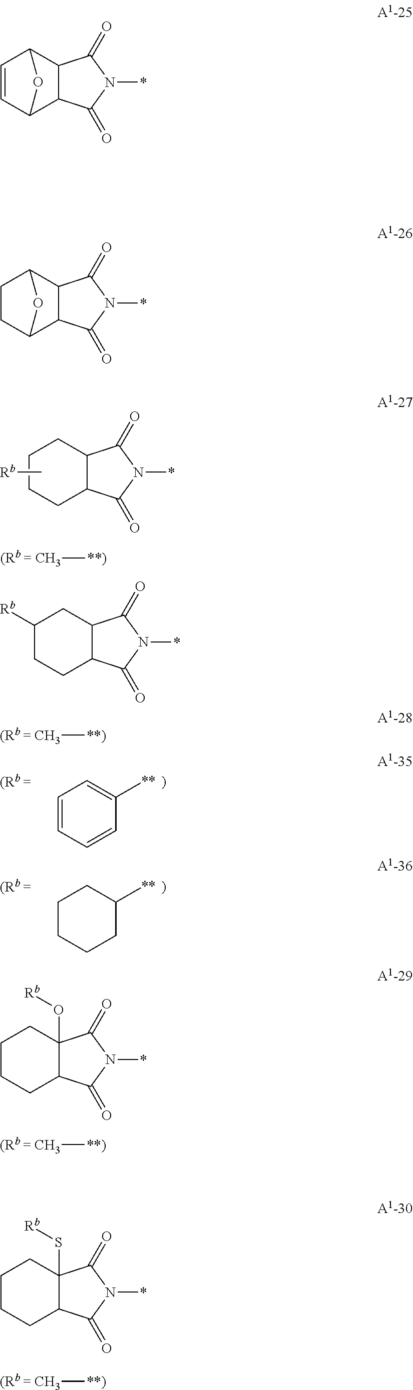

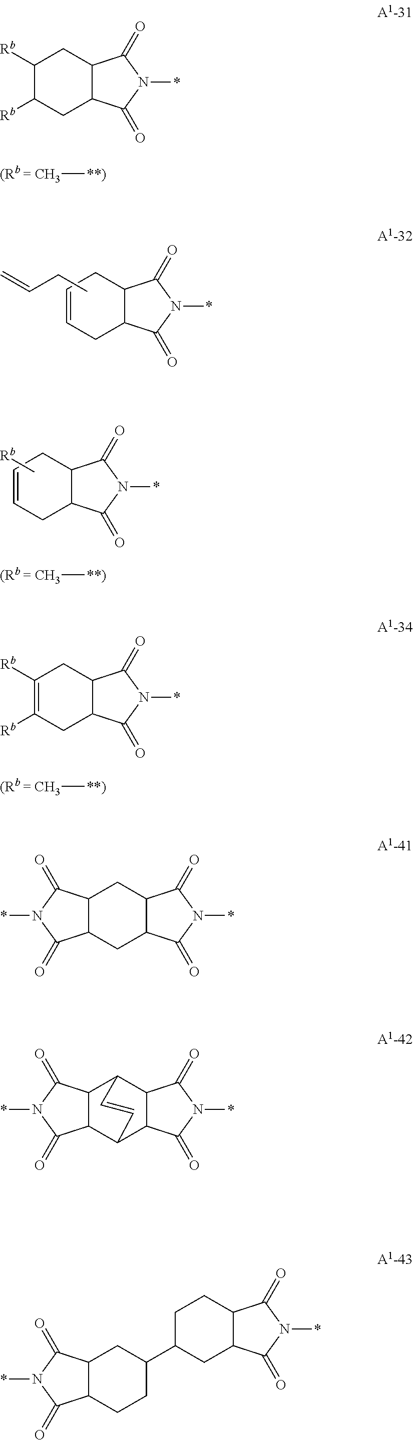

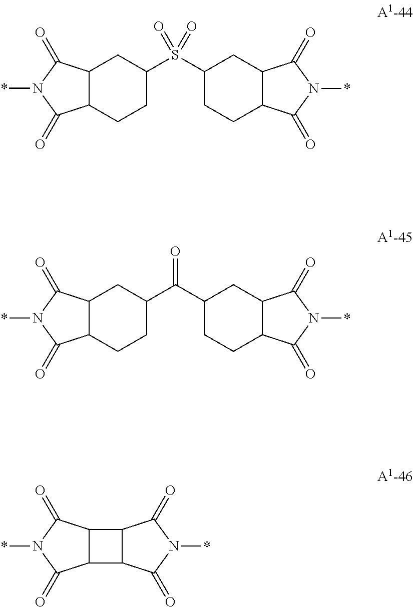 [SMILES: *N1C(=O)C2CCC(S(=O)(=O)C3CCC4C(=O)N(*)C(=O)C4C3)CC2C1=O ; *N1C(=O)C2CCC(C(=O)C3CCC4C(=O)N(*)C(=O)C4C3)CC2C1=O ; *N1C(=O)C2C(C1=O)C1C(=O)N(*)C(=O)C21]

Among these, A¹-21 to A¹-28, A¹-31, and A¹-33 to A¹-34 are more preferable.

<<Formula (III)>>

A² represents a group represented by Formula (III).

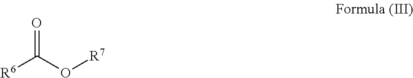

(In Formula (III), R⁶ and R⁷ each independently represent a substituted or unsubstituted phenyl group or a substituted or unsubstituted alkyl group. Here, at least one of R⁶ and R⁷ represents a bonding site with L in Formula (I).)

R⁶ and R⁷ each independently represent a substituted or unsubstituted phenyl group or a substituted or unsubstituted alkyl group. As the substituted or unsubstituted alkyl group, an alkyl group having 1 to 15 carbon atoms is preferable, an alkyl group having 1 to 10 carbon atoms is more preferable, and an alkyl group having 1 to 6 carbon atoms is still more preferable.

Examples of a substituent which may be included in a phenyl group and an alkyl group include the substituent group T.

As a substituent of the phenyl group, an alkyl group (preferably a methyl group), an alkoxycarbonyl group, an acyl group, acyloxy group, or an alkoxy group is preferable, and an unsubstituted phenyl group is more preferable.

As a substituent of the alkyl group, an alkoxycarbonyl group, an acyl group, an acyloxy group, an alkoxy group, a hydroxyl group, or a cyano group is preferable, and a cyano group or an unsubstituted alkyl group is more preferable.

In a case where there are a plurality of groups represented by Formula (III), the groups may be bonded to each other to form a ring. For example, there is a mode (for example, the following A²-20, A²-21, A²-22, A²-23, A²-24, or A²-25) in which two R⁶s are bonded to each other to form a benzene ring or a cyclohexane ring. It is possible to reduce the free volume existing in cellulose acylate through interaction between the cellulose acylate and a carbonyl group in an ester bond which is contained in a group represented by Formula (III) in addition to a carbonyl group in an imide bond which is contained in a group represented by Formula (II), in the cellulose acylate. Therefore, it is possible to contribute to the improvement of hardness of the film.

The group represented by Formula (III) may be mixed with a geometric isomer or an enantiomer.

At least one of R⁶ and R⁷ represents a bonding site with L, and one of R⁶ and R⁷ is preferably bonded to L and R⁷ is more preferably bonded to L.

Specifically, the group represented by Formula (III) is preferably a group exemplified below. ** represents a position which is bonded to L and *** represents a position which is bonded to a substituent R^{a} or R^{b} (R^{a} represents an alkyl group, R^{b} represents an alkyl group, a cyclic alkyl group, or an aryl group).

As the alkyl group represented by R^{a} or R^{b}, an alkyl group having 1 to 10 carbon atoms is preferable, an alkyl group having 1 to 5 carbon atoms is more preferable, and an alkyl group having 1 to 3 carbon atoms is still more preferable.

As the cyclic alkyl group represented by R^{b}, a cyclic alkyl group having 6 to 12 carbon atoms is preferable, a cyclic alkyl group having 6 to 8 carbon atoms is more preferable, and a cyclic alkyl group having 6 to 8 carbon atoms is still more preferable.

As the aryl group represented by R^{b}, an aryl group having 6 to 12 carbon atoms is preferable, an aryl group having 6 to 10 carbon atoms is more preferable, and an aryl group having 6 to 8 carbon atoms is still more preferable.

These groups may be straight-chain, branched, or cyclic groups. Examples of a substituent which may be included in an alkyl group, a cyclic alkyl group, ./ and an aryl group include the substituent group T. Among these, an alkyl group, an alkenyl group, an aryl group, an alkoxy group, an aryloxy group, an acyl group, an alkoxycarbonyl group, an acyloxy group, an acylamino group, an alkoxycarbonylamino group, a carbamoyl group, an alkylthio group, a ureido group, a hydroxy group, a cyano group, a carboxyl group, or a silyl group is preferable.

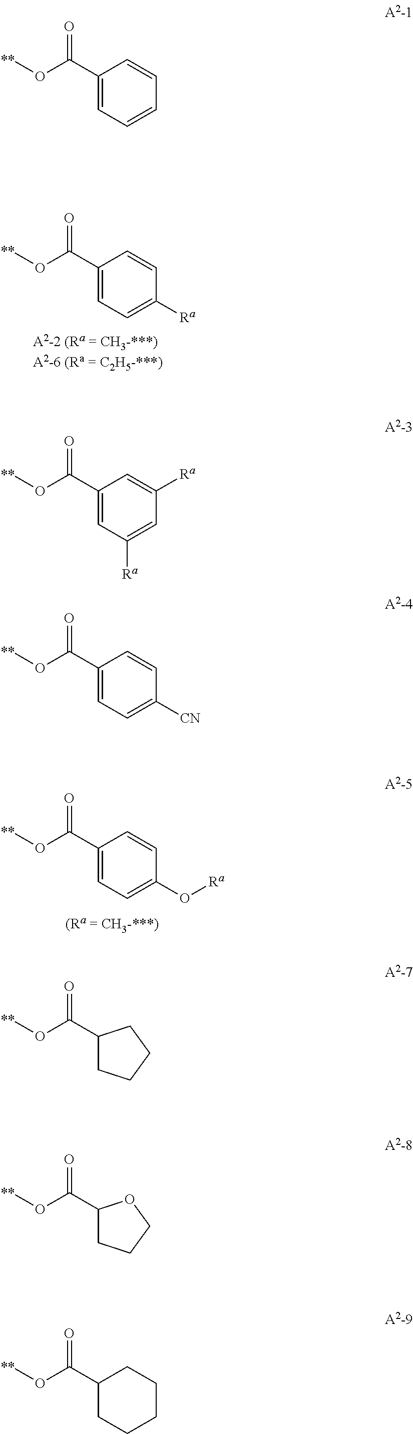

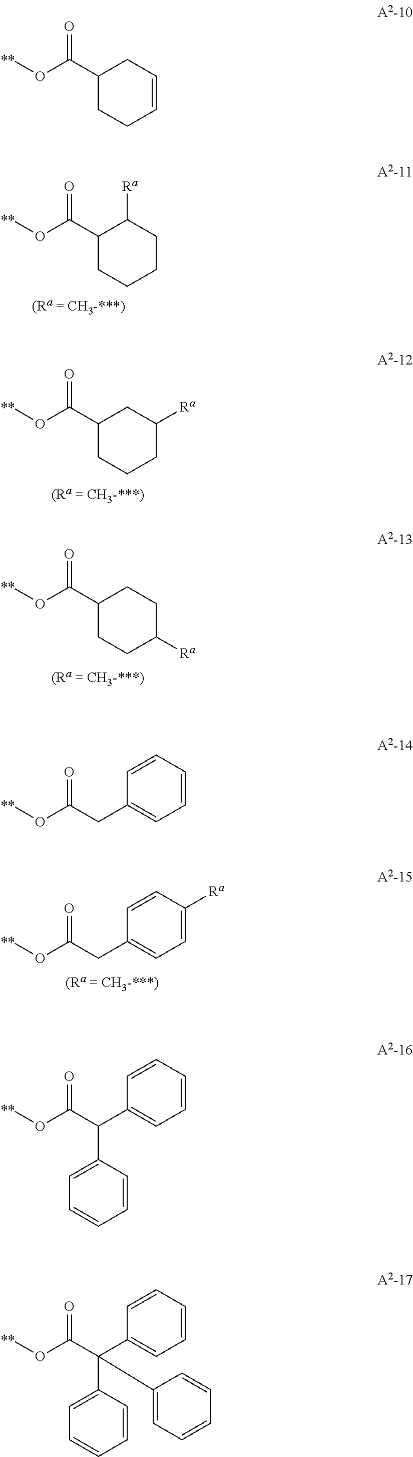

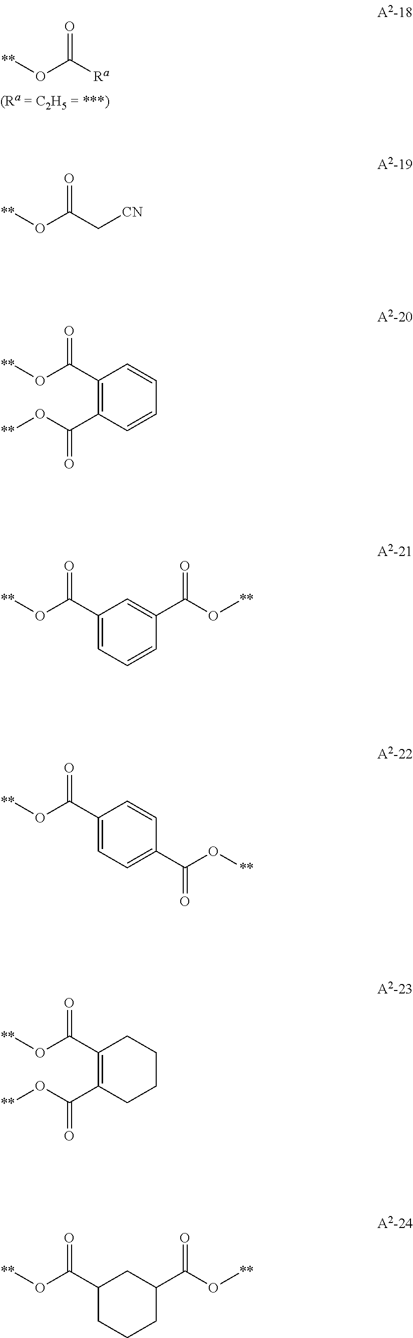

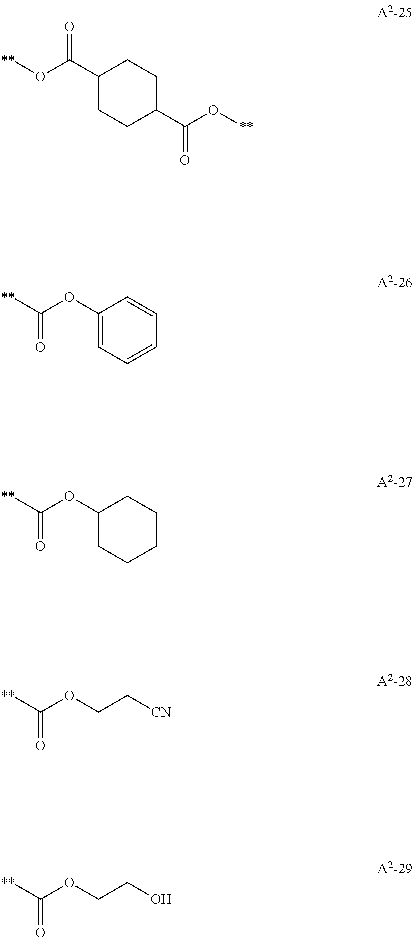

Among these, A²-1 to A²-3, A²-6, A²-7, A²-9, A²-11 to A²-16, A²-19, A²-20 to A²-25, and A²-27 to A²-29 are more preferable.

<<Preferred Mode of Compound Represented by Formula (I)>>

The compound represented by Formula (I) is preferably a compound represented by the following Formula (I-1).

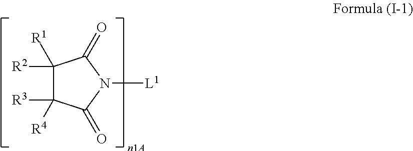

(In Formula (I-1), L¹ represents a linking group of (n1A) valence of which the number of atoms is less than or equal to 8. n1A represents an integer of 2 to 4. R¹ to R⁴ each independently represent a hydrogen atom, a substituted or unsubstituted alkyl group, a substituted or unsubstituted acyl group, a substituted or unsubstituted alkoxycarbonyl group, a substituted or unsubstituted alkoxy group, a substituted or unsubstituted aryloxy group, a substituted or unsubstituted alkylthio group, a substituted or unsubstituted arylthio group, a substituted or unsubstituted carbamoyl group, a carboxyl group, a cyano group, or a hydroxyl group, and R¹ or R² and R³ or R⁴ may be bonded to each other to form a non-aromatic ring.)

The compound represented by Formula (I-1) is in a mode in which n1 in Formula (I) is an integer of 2 to 4 and n2 is 0.

Regarding L¹ in Formula (I-1), it is possible to refer to the disclosure corresponding to the divalent to tetravalent groups in the description about L in Formula (I), and a preferred range of L¹ in Formula (I-1) is the same as that of L in Formula (I).

R¹ to R⁴ in Formula (I-1) are respectively synonymous with R¹ to R⁴ in Formula (II), and preferred ranges of R¹ to R⁴ in Formula (I-1) are the same as those of R¹ to R⁴ in Formula (II).

n1A represents an integer of 2 to 4, and is preferably an integer of 2 or 3 and more preferably an integer of 2.

The compound represented by Formula (I) is preferably a compound represented by the following Formula (I-2).

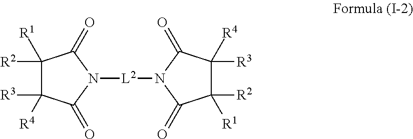

(In Formula (I-2), L² represents a divalent linking group of which the number of atoms linking a nitrogen atom to a nitrogen atom is less than or equal to 8. R¹ to R⁴ each independently represent a hydrogen atom, a substituted or unsubstituted alkyl group, a substituted or unsubstituted acyl group, a substituted or unsubstituted alkoxycarbonyl group, a substituted or unsubstituted alkoxy group, a substituted or unsubstituted aryloxy group, a substituted or unsubstituted alkylthio group, a substituted or unsubstituted arylthio group, a substituted or unsubstituted carbamoyl group, a carboxyl group, a cyano group, or a hydroxyl group, and R¹ or R² and R³ or R⁴ may be bonded to each other to form a non-aromatic ring).

The compound represented by Formula (I-2) is in a mode in which n1 in Formula (I) is 2 and n2 is 0.

Regarding L² in Formula (I-2), it is possible to refer to the description corresponding to the divalent group in the description about L in Formula (I), and a preferred range of L² in Formula (I-2) is the same as that of L in Formula (I).

R¹ to R⁴ in Formula (I-2) are respectively synonymous with R¹ to R⁴ in Formula (II), and preferred ranges of R¹ to R⁴ in Formula (I-2) are the same as those of R¹ to R⁴ in Formula (II).

The compound represented by Formula (I) is preferably a compound represented by the following Formula (I-3).

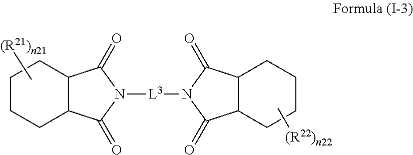

(In Formula (I-3), L³ represents a divalent linking group which contains at least one of a tertiary carbon atom and a quaternary carbon atom and of which the number of carbon atoms linking a nitrogen atom to a nitrogen atom is less than or equal to 8. R²¹ and R²² represent a methyl group. n21 and n22 each represent an integer of 0 or 1.)

L³ represents a divalent linking group which contains at least one of a tertiary carbon atom and a quaternary carbon atom and of which the number of carbon atoms linking a nitrogen atom to a nitrogen atom is less than or equal to 8. L³ may be only one kind of a straight-chain, branched, or cyclic aliphatic group and aromatic group, and examples of the group containing a branched or cyclic aliphatic group include a 1-methyl ethylene group, a 1,3-cyclohexylene group, and a 1,2-cyclohexylene group.

Specifically, L³ is preferably a structure shown below (* represents a bonding site with a nitrogen atom).

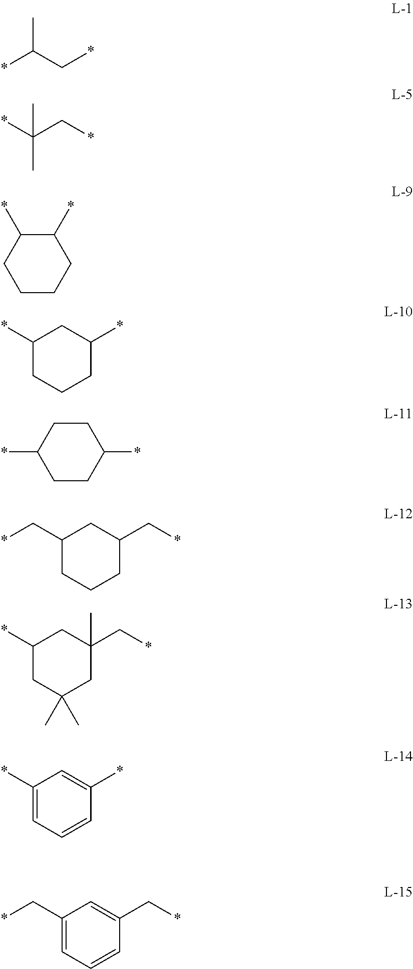

As n21 and n22, 0 is preferable.

The compound represented by Formula (I) is preferably a compound represented by the following Formula (I-4).

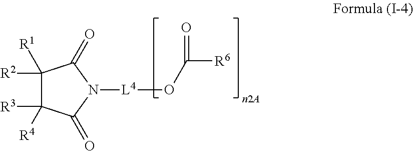

(In Formula (I-4), L⁴ represents a linking group of (n2A+1) valence of which the number of atoms linking a nitrogen atom to an oxygen atom is less than or equal to 8. n2A represents an integer of 1 to 4. R¹ to R⁴ each independently represent a hydrogen atom, a substituted or unsubstituted alkyl group, a substituted or unsubstituted acyl group, a substituted or unsubstituted alkoxycarbonyl group, a substituted or unsubstituted alkoxy group, a substituted or unsubstituted aryloxy group, a substituted or unsubstituted alkylthio group, a substituted or unsubstituted arylthio group, a substituted or unsubstituted carbamoyl group, a carboxyl group, a cyano group, or a hydroxyl group, and R¹ or R² and R³ or R⁴ may be bonded to each other to form a non-aromatic ring. R⁶ represents a substituted or unsubstituted phenyl group or a substituted or unsubstituted alkyl group.)

Regarding L⁴ in Formula (I-4), it is possible to refer to the disclosure corresponding to the divalent to tetravalent groups in the description about L in Formula (I), and a preferred range of L⁴ in Formula (I-4) is the same as that of L in Formula (I).

R¹ to R⁴ in Formula (I-4) are respectively synonymous with R¹ to R⁴ in Formula (II), and preferred ranges of R¹ to R⁴ in Formula (I-4) are the same as those of R¹ to R⁴ in Formula (II).

R⁶ in Formula (I-4) is synonymous with R⁶ in Formula (III), and a preferred range of R⁶ in Formula (I-4) is the same as that of R⁶ in Formula (III).

n2A represents an integer of 1 to 4, and is preferably an integer of 1 to 3.

The compound represented by Formula (I) is preferably a compound represented by the following Formula (I-5).

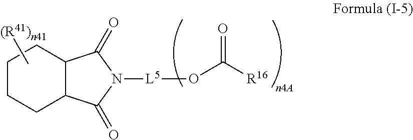

(In Formula (I-5), L⁵ represents a linking group selected from the following linking group A, R⁴¹ represents a methyl group, and n41 represents an integer of 0 or 1. R¹⁶ represents a linking group selected from the following substituent group B, and a plurality of the substituents may be the same as or different from each other. n4A represents an integer of 1 to 3.)

### Linking Group A:

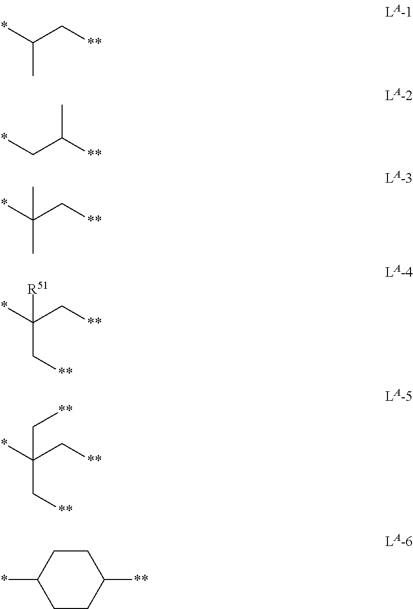

(* represents a bonding site with a nitrogen atom and ** represents a bonding site with an oxygen atom. R⁵¹ represents a hydrogen atom, a methyl group, or an ethyl group.)

Substituent group B:

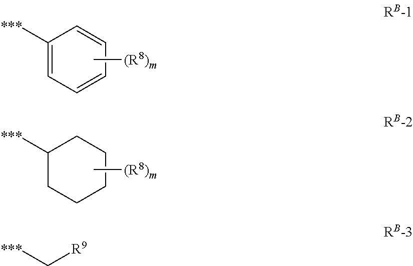

(*** represents a bonding site with a carbonyl group, R⁸ represents an alkyl group, an alkoxycarbonyl group, an acyl group, or an alkoxy group, and R⁹ represents a carbonyl group, an alkoxycarbonyl group, a carbonyloxy group, a cyano group, or a hydroxyl group. m represents an integer of 0 to 3.)

L⁵ represents a linking group selected from the above-described linking group A, and among the above-described linking group A, L^{A}-1 to L^{A}-5 are more preferable.

R⁵¹ represents a hydrogen atom, a methyl group, or an ethyl group, and a hydrogen atom is more preferable.

R¹⁶ represents a substituent selected from the above-described substituent group B, and among the above-described substituent group B, R^{B}-1 is preferable.

R⁸ represents an alkyl group, an alkoxycarbonyl group, an acyl group, and an alkoxy group, and is more preferably an alkyl group.

Here, the alkyl group is preferably a methyl group, an ethyl group, or a propyl group. The alkoxycarbonyl group is preferably a methoxycarbonyl group or an ethoxycarbonyl group. The acyl group is preferably an acetyl group. The alkoxy group is preferably a methoxy group or an ethoxy group.

Examples of R⁹ include a carbonyl group, an alkoxycarbonyl group, a carbonyloxy group, a cyano group, or a hydroxyl group, and among these, a cyano group is more preferable.

m represents an integer of 0 to 3, and is preferably an integer of 0 to 2 and more preferably 0.

n4A represents an integer of 1 to 3.

Hereinafter, the compound represented by Formula (I) which is preferably used in the present invention will be exemplified, but the present invention is not limited thereto.

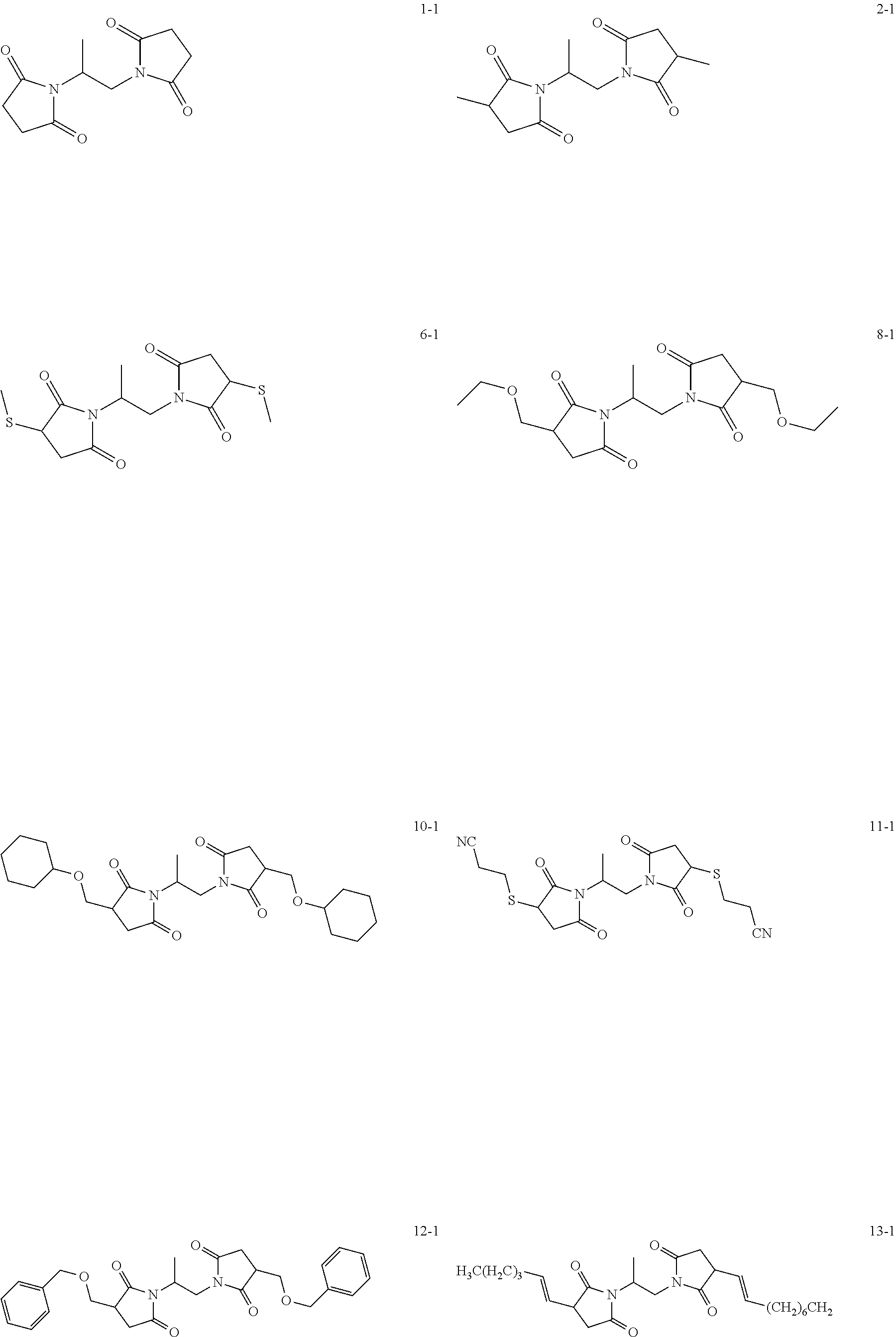

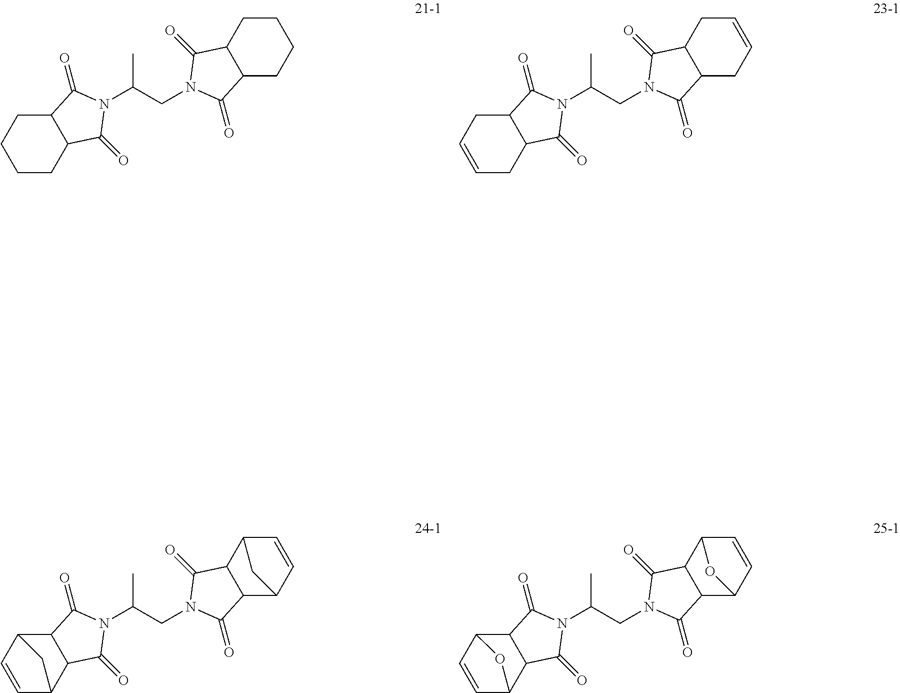 [SMILES: CC(CN1C(=O)C2CCCCC2C1=O)N1C(=O)C2CCCCC2C1=O ; CC(CN1C(=O)C2CC=CCC2C1=O)N1C(=O)C2CC=CCC2C1=O ; CC(CN1C(=O)C2C3C=CC(C3)C2C1=O)N1C(=O)C2C3C=CC(C3)C2C1=O ; CC(CN1C(=O)C2C3C=CC(O3)C2C1=O)N1C(=O)C2C3C=CC(O3)C2C1=O]

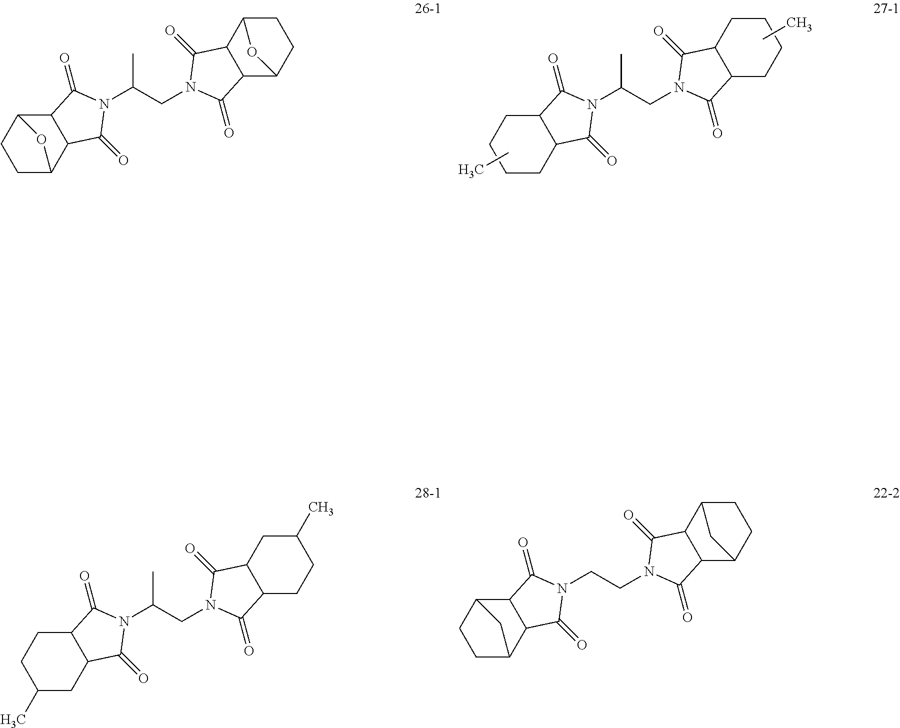

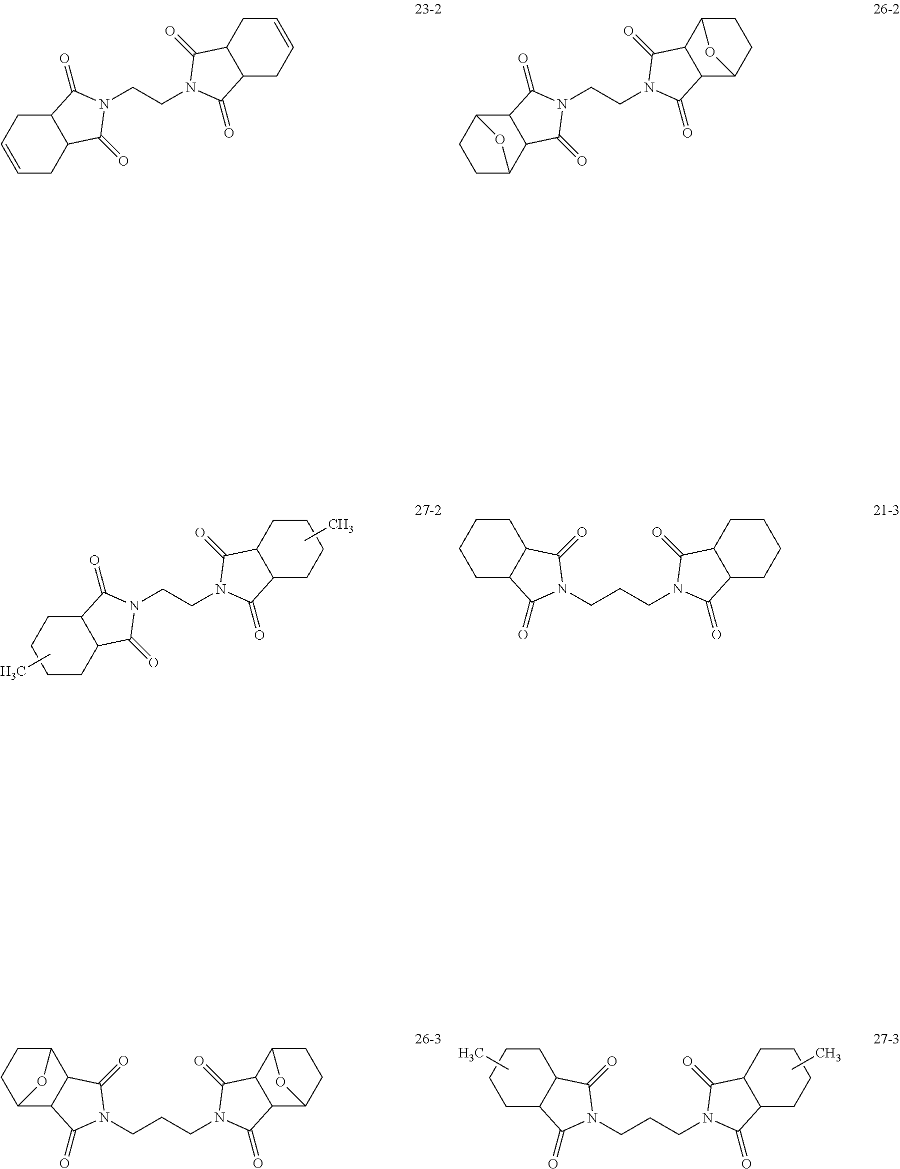

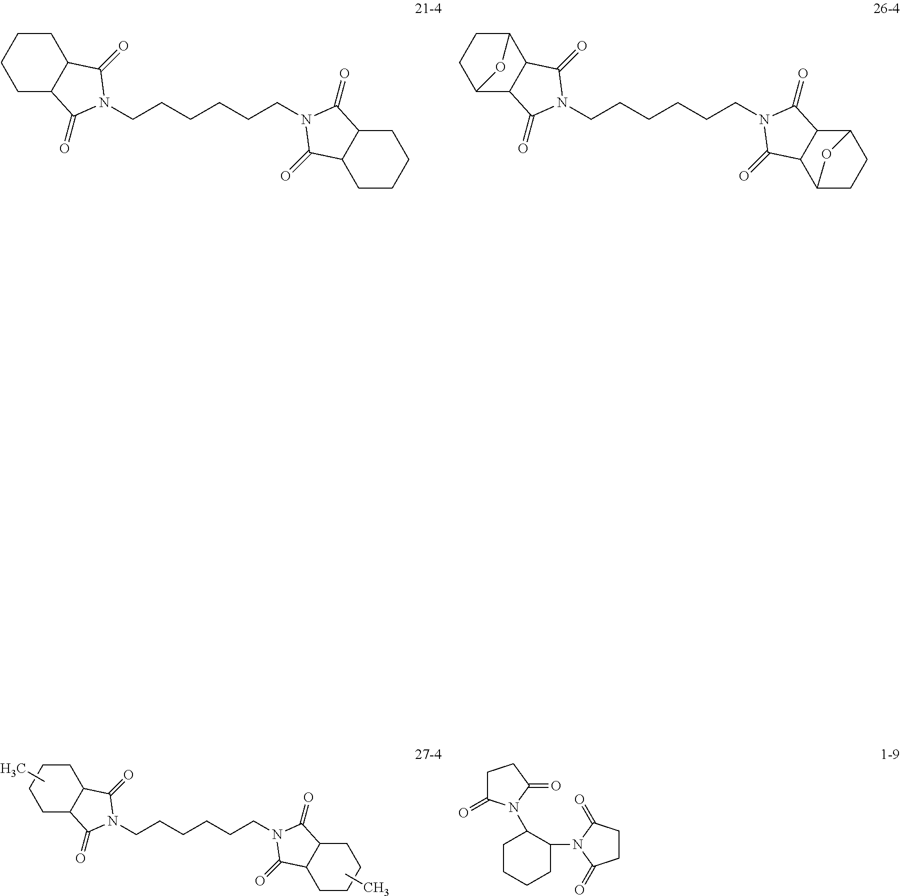

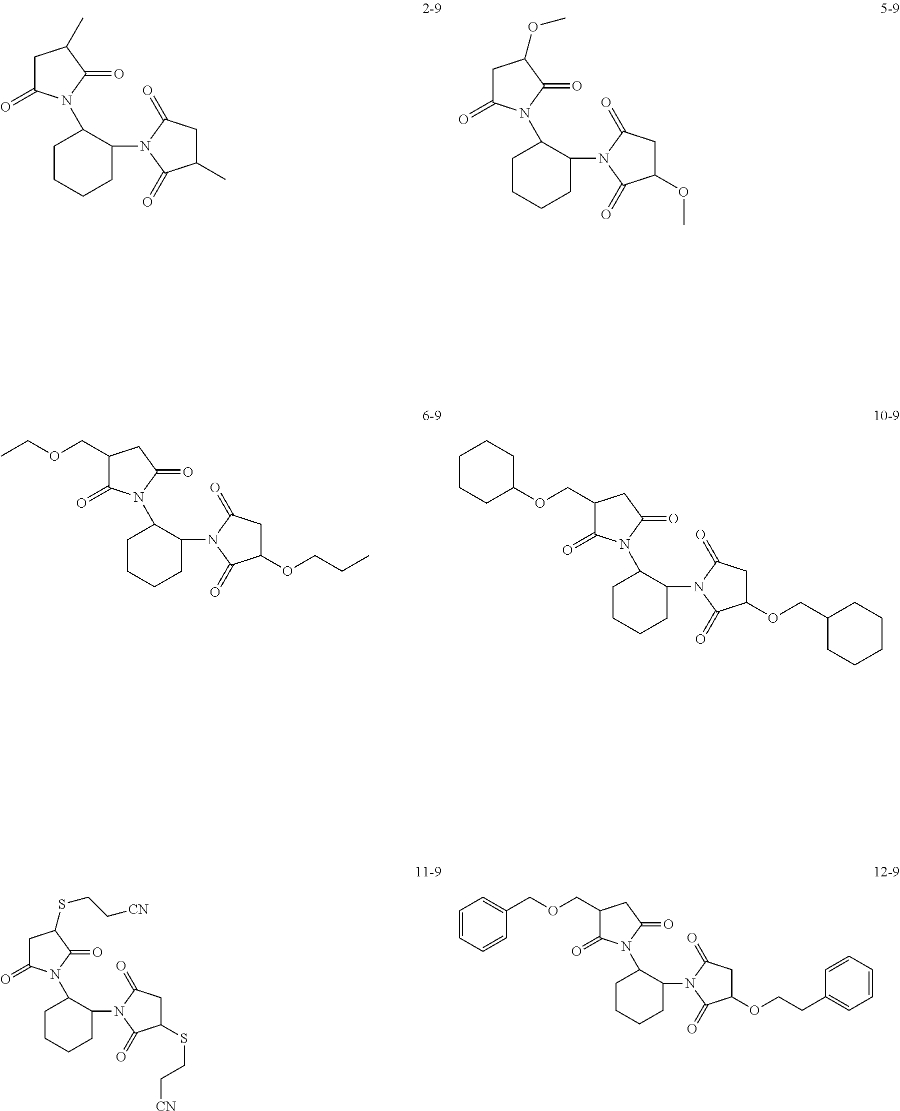 [SMILES: CC1CC(=O)N(C2CCCCC2N2C(=O)CC(C)C2=O)C1=O ; COC1CC(=O)N(C2CCCCC2N2C(=O)CC(OC)C2=O)C1=O ; CCCOC1CC(=O)N(C2CCCCC2N2C(=O)CC(COCC)C2=O)C1=O ; O=C1CC(COC2CCCCC2)C(=O)N1C1CCCCC1N1C(=O)CC(OCC2CCCCC2)C1=O ; N#CCCSC1CC(=O)N(C2CCCCC2N2C(=O)CC(SCCC#N)C2=O)C1=O ; O=C1CC(COCc2ccccc2)C(=O)N1C1CCCCC1N1C(=O)CC(OCCc2ccccc2)C1=O]

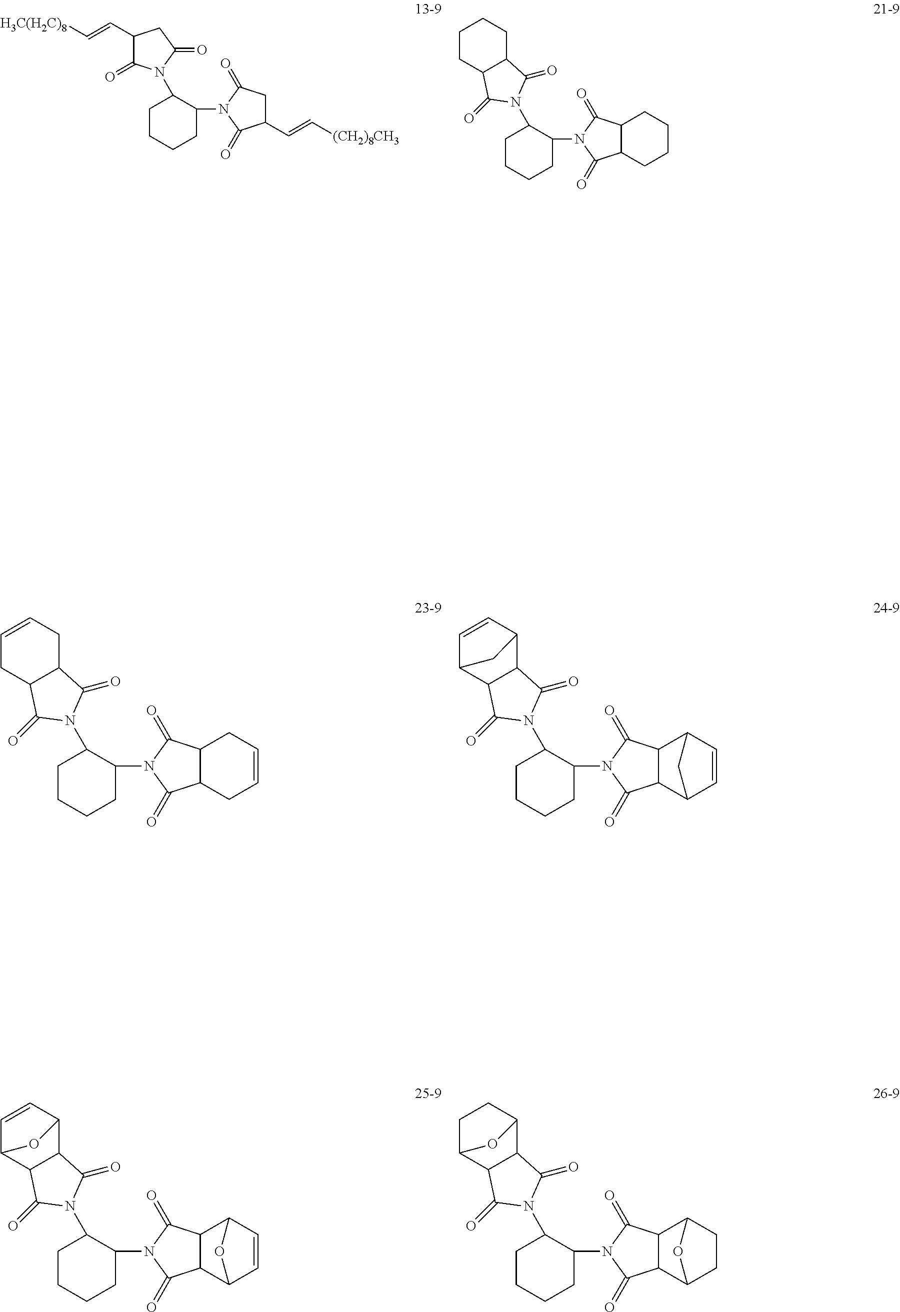

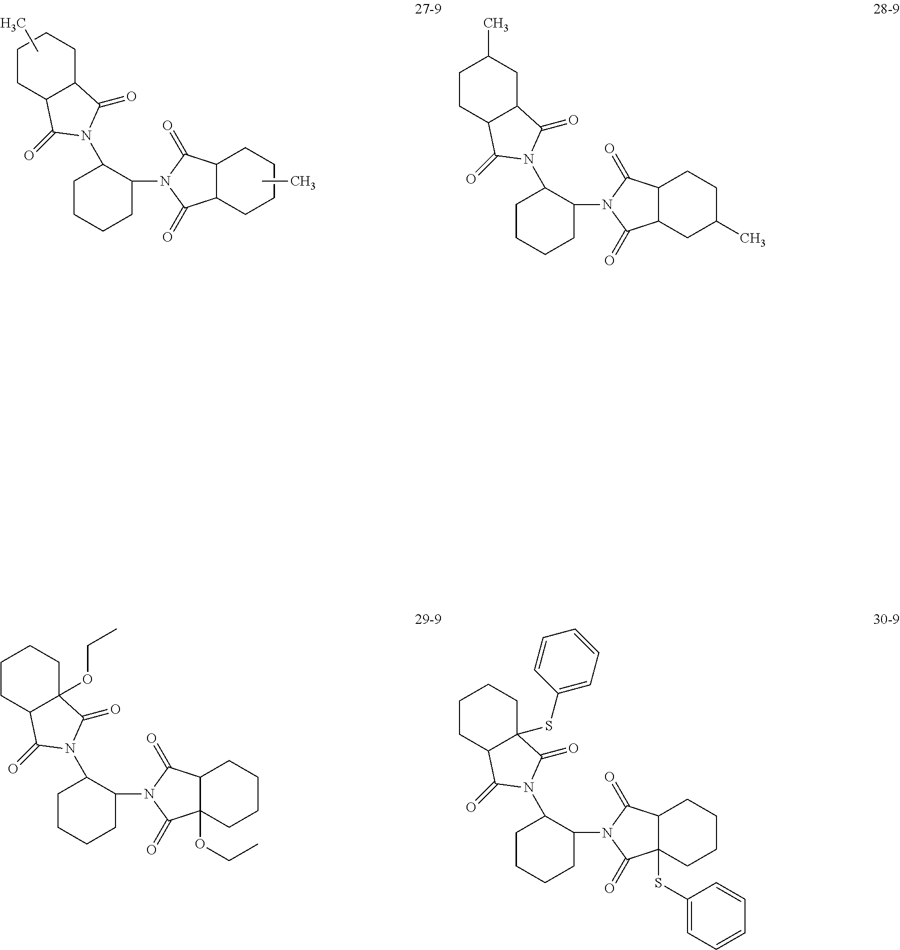

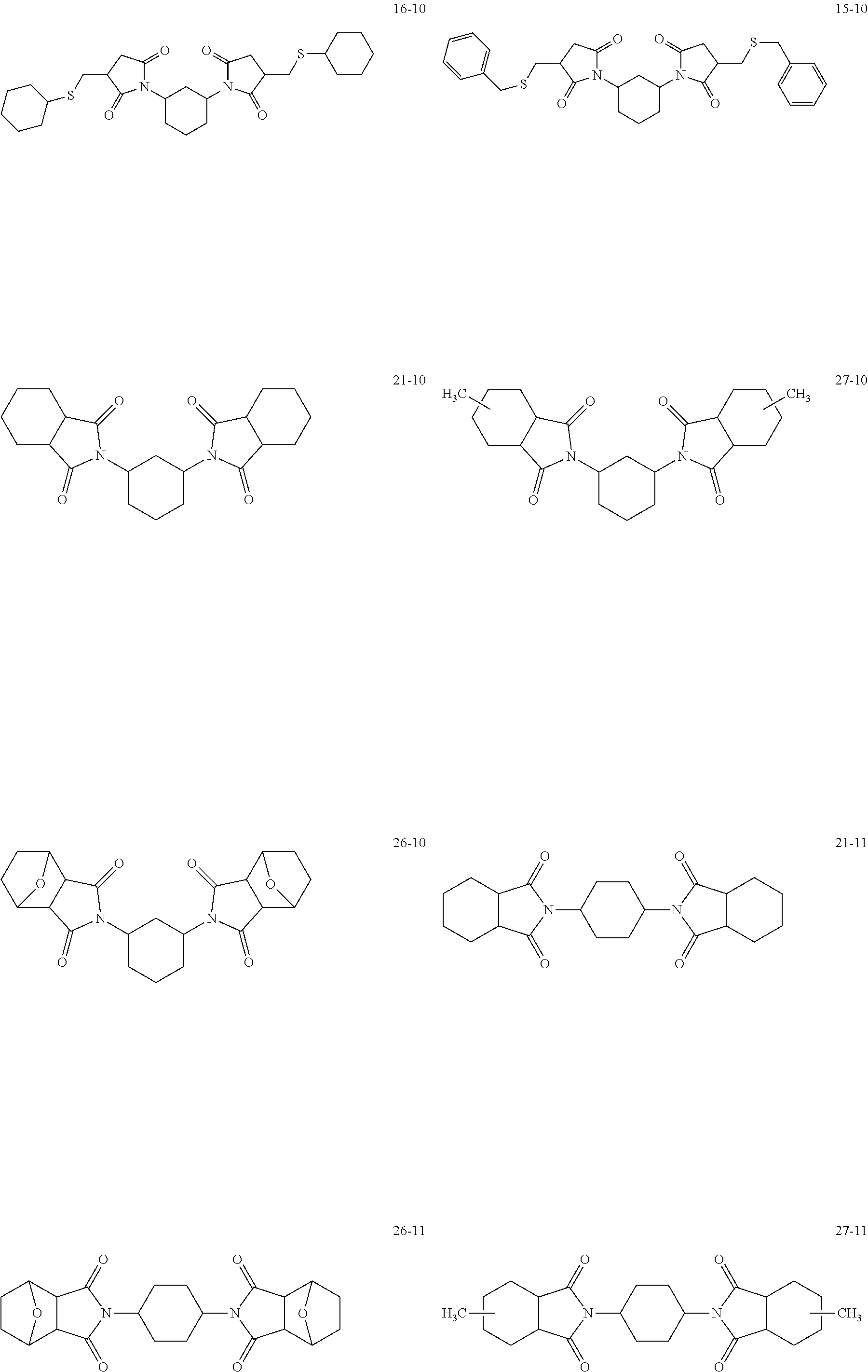

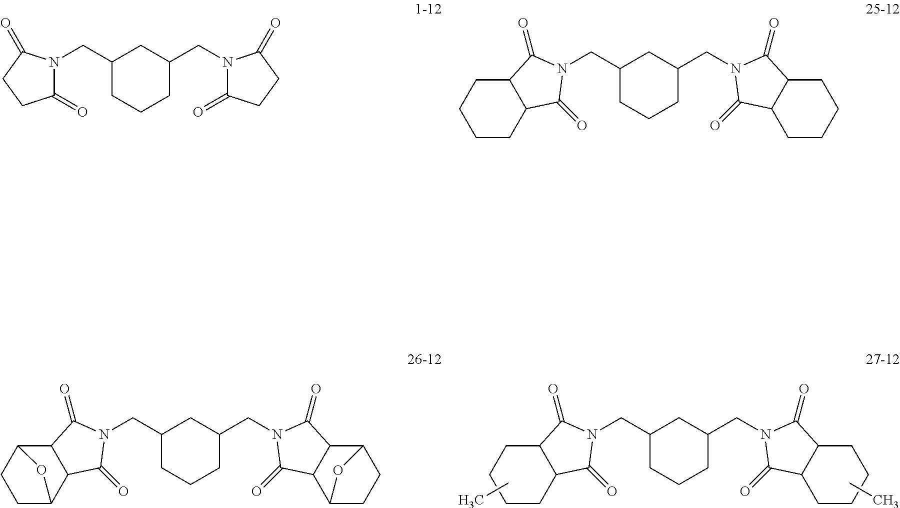

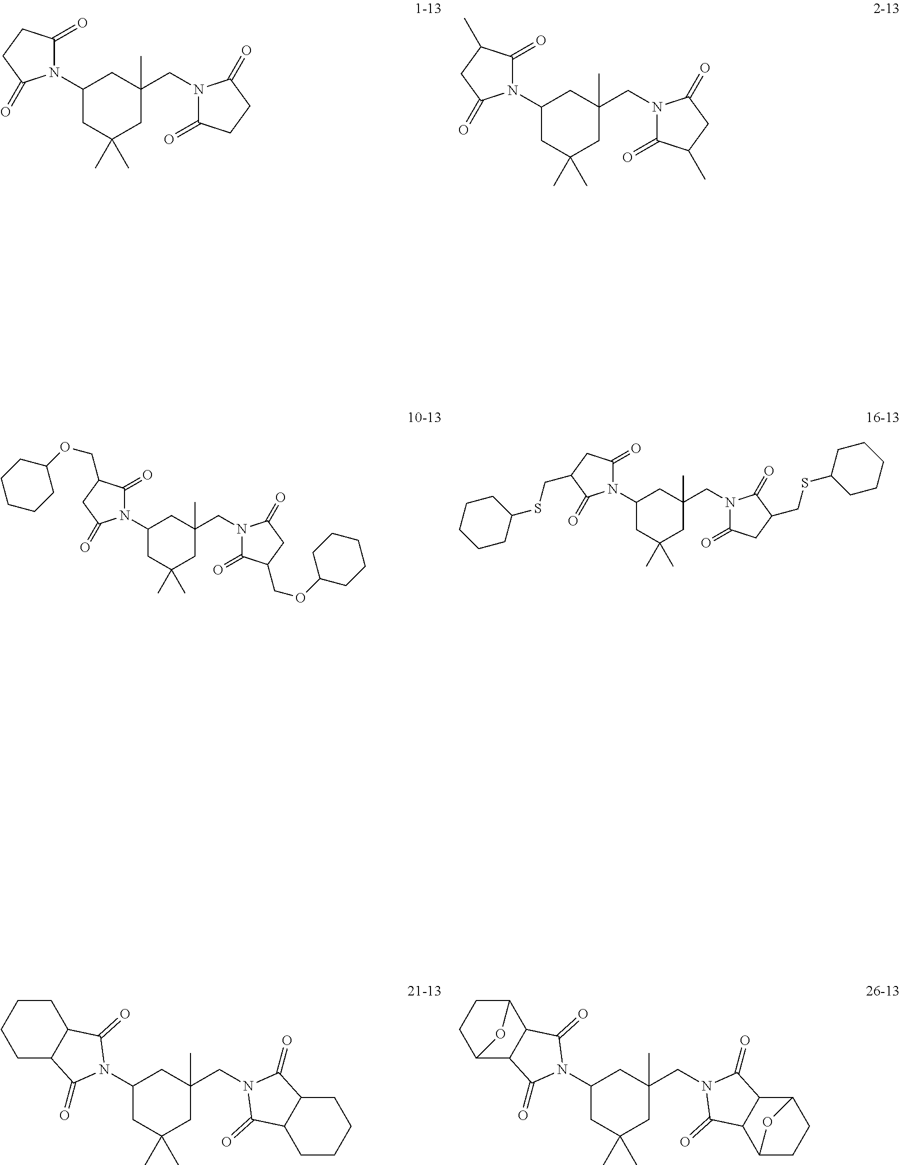 [SMILES: CC1(C)CC(N2C(=O)CCC2=O)CC(C)(CN2C(=O)CCC2=O)C1 ; CC1CC(=O)N(CC2(C)CC(N3C(=O)CC(C)C3=O)CC(C)(C)C2)C1=O ; CC1(C)CC(N2C(=O)CC(COC3CCCCC3)C2=O)CC(C)(CN2C(=O)CC(COC3CCCCC3)C2=O)C1 ; CC1(C)CC(N2C(=O)CC(CSC3CCCCC3)C2=O)CC(C)(CN2C(=O)CC(CSC3CCCCC3)C2=O)C1 ; CC1(C)CC(N2C(=O)C3CCCCC3C2=O)CC(C)(CN2C(=O)C3CCCCC3C2=O)C1 ; CC1(C)CC(N2C(=O)C3C4CCC(O4)C3C2=O)CC(C)(CN2C(=O)C3C4CCC(O4)C3C2=O)C1]

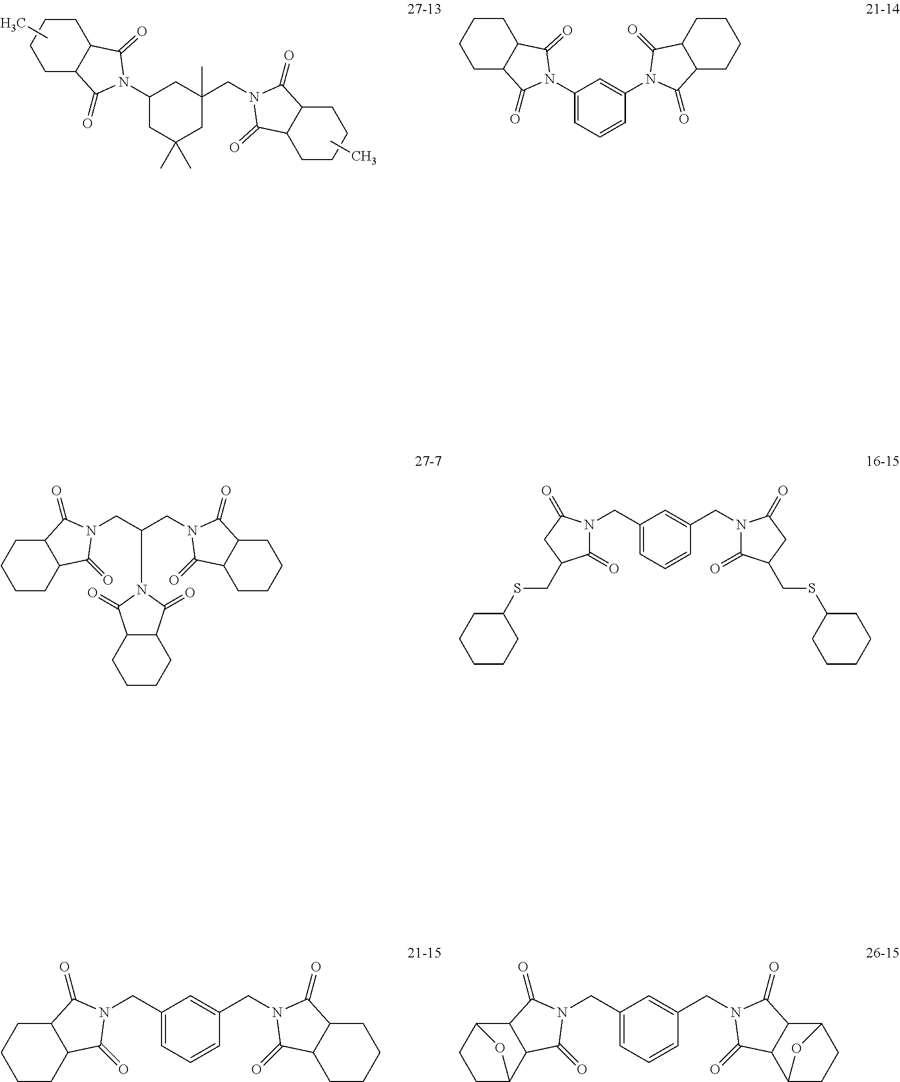

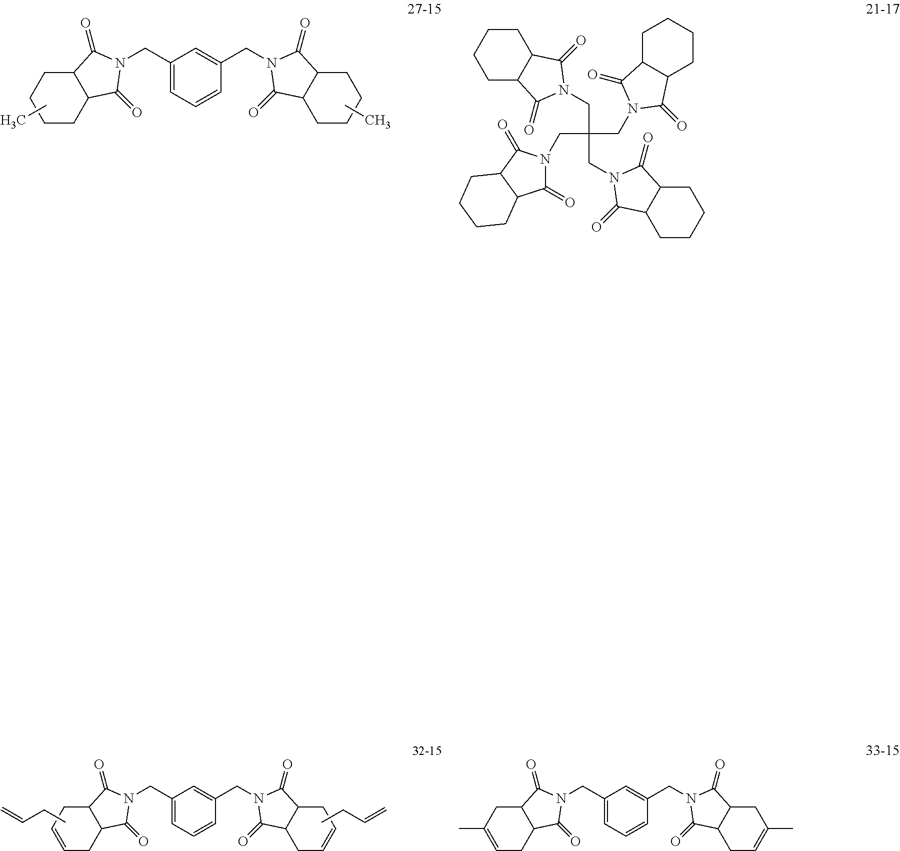

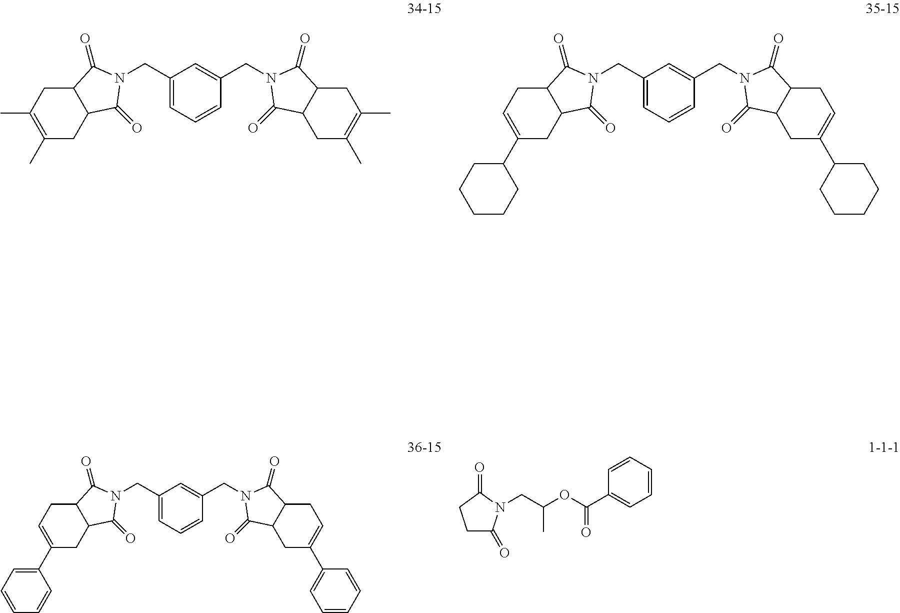

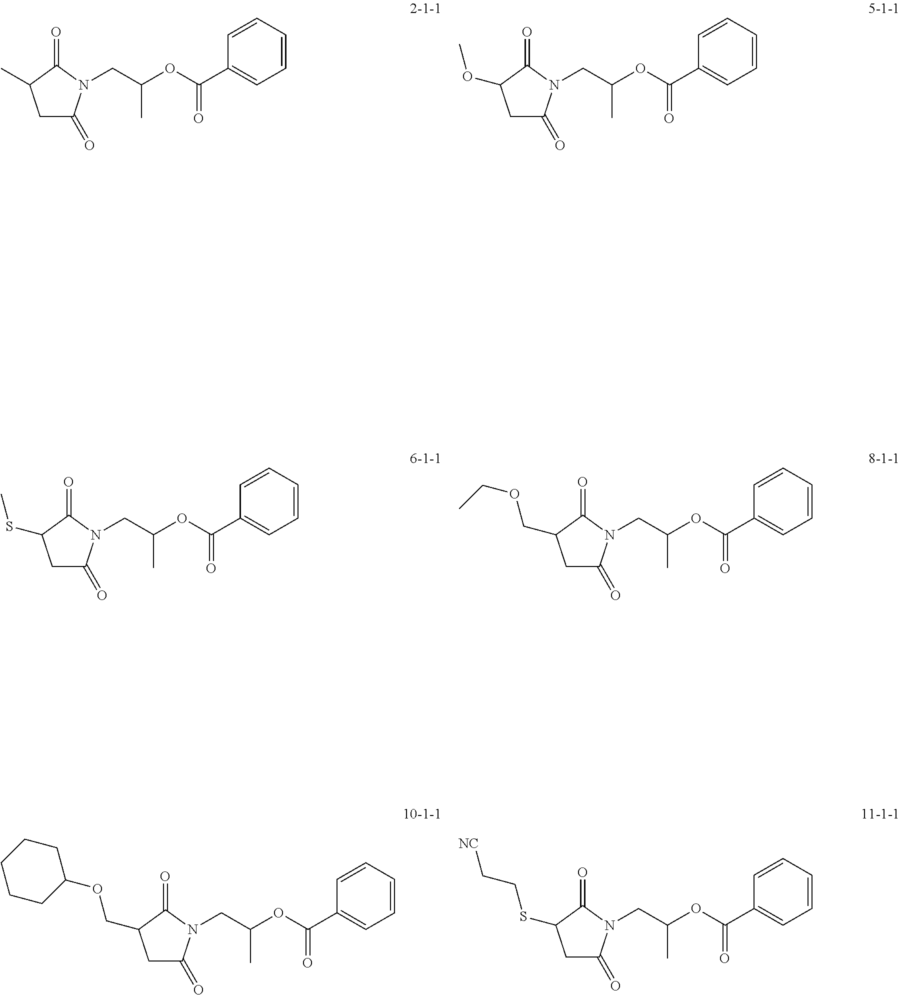 [SMILES: CC(CN1C(=O)CC(C)C1=O)OC(=O)c1ccccc1 ; COC1CC(=O)N(CC(C)OC(=O)c2ccccc2)C1=O ; CSC1CC(=O)N(CC(C)OC(=O)c2ccccc2)C1=O ; CCOCC1CC(=O)N(CC(C)OC(=O)c2ccccc2)C1=O ; CC(CN1C(=O)CC(COC2CCCCC2)C1=O)OC(=O)c1ccccc1 ; CC(CN1C(=O)CC(SCCC#N)C1=O)OC(=O)c1ccccc1]

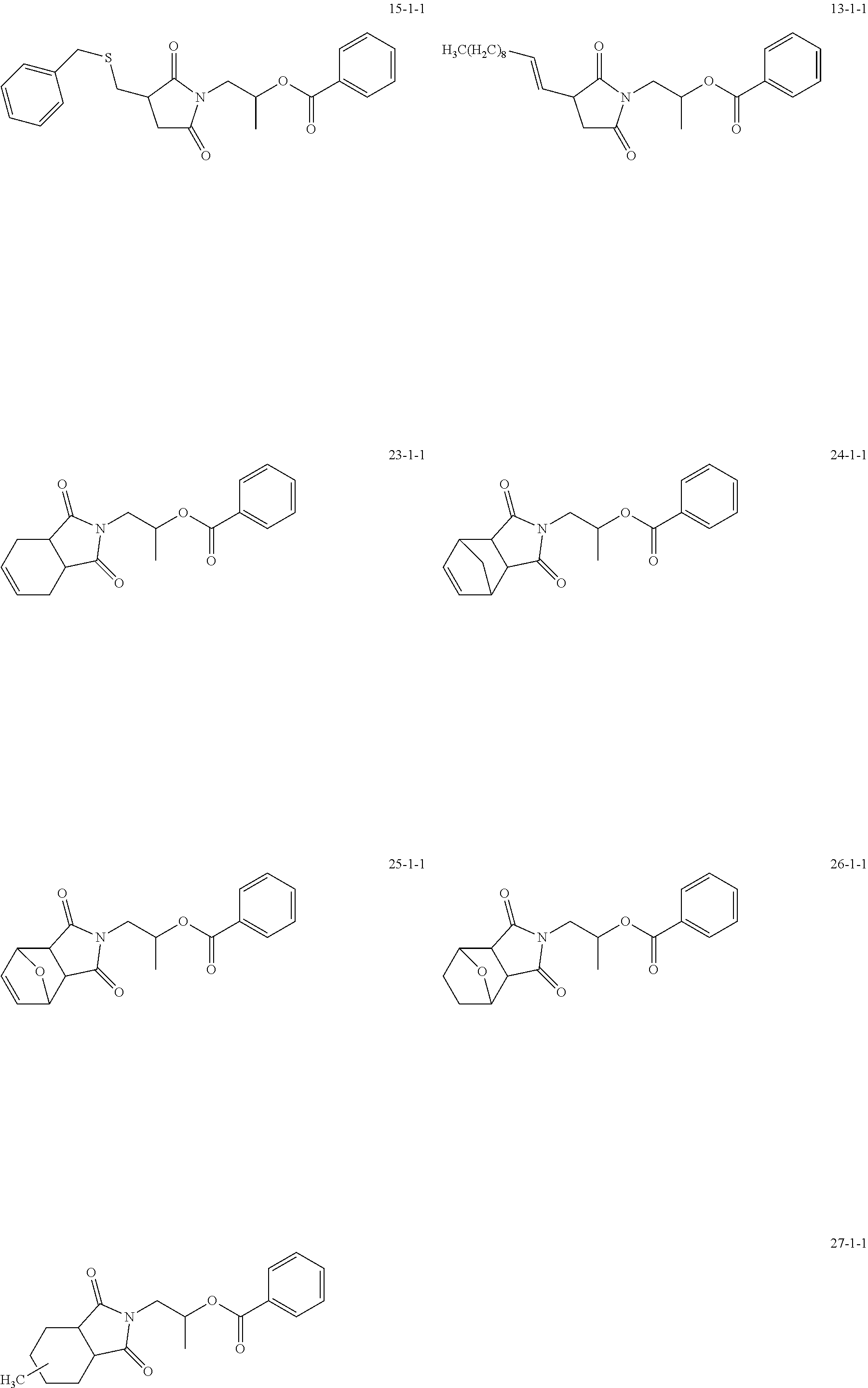

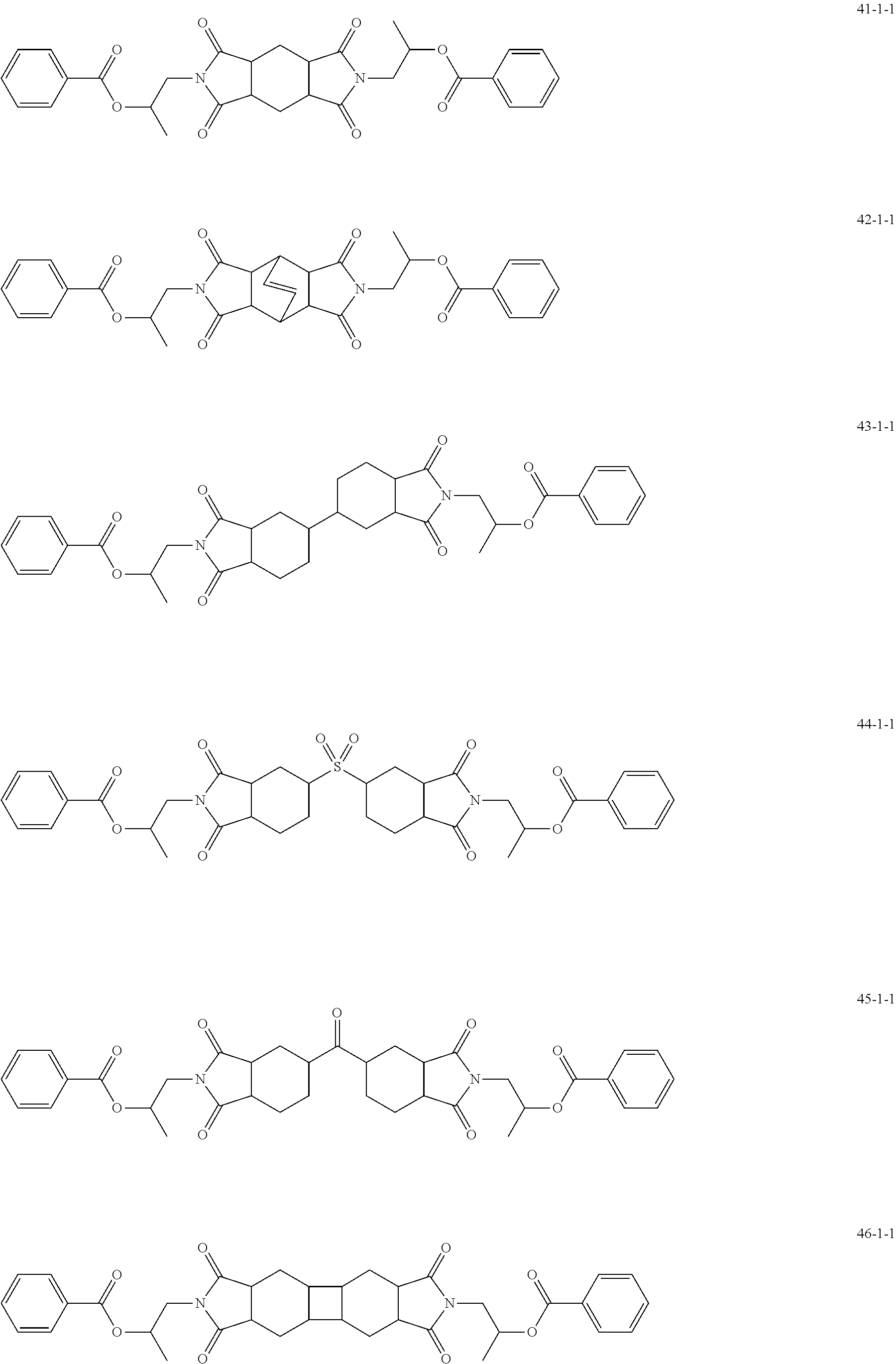

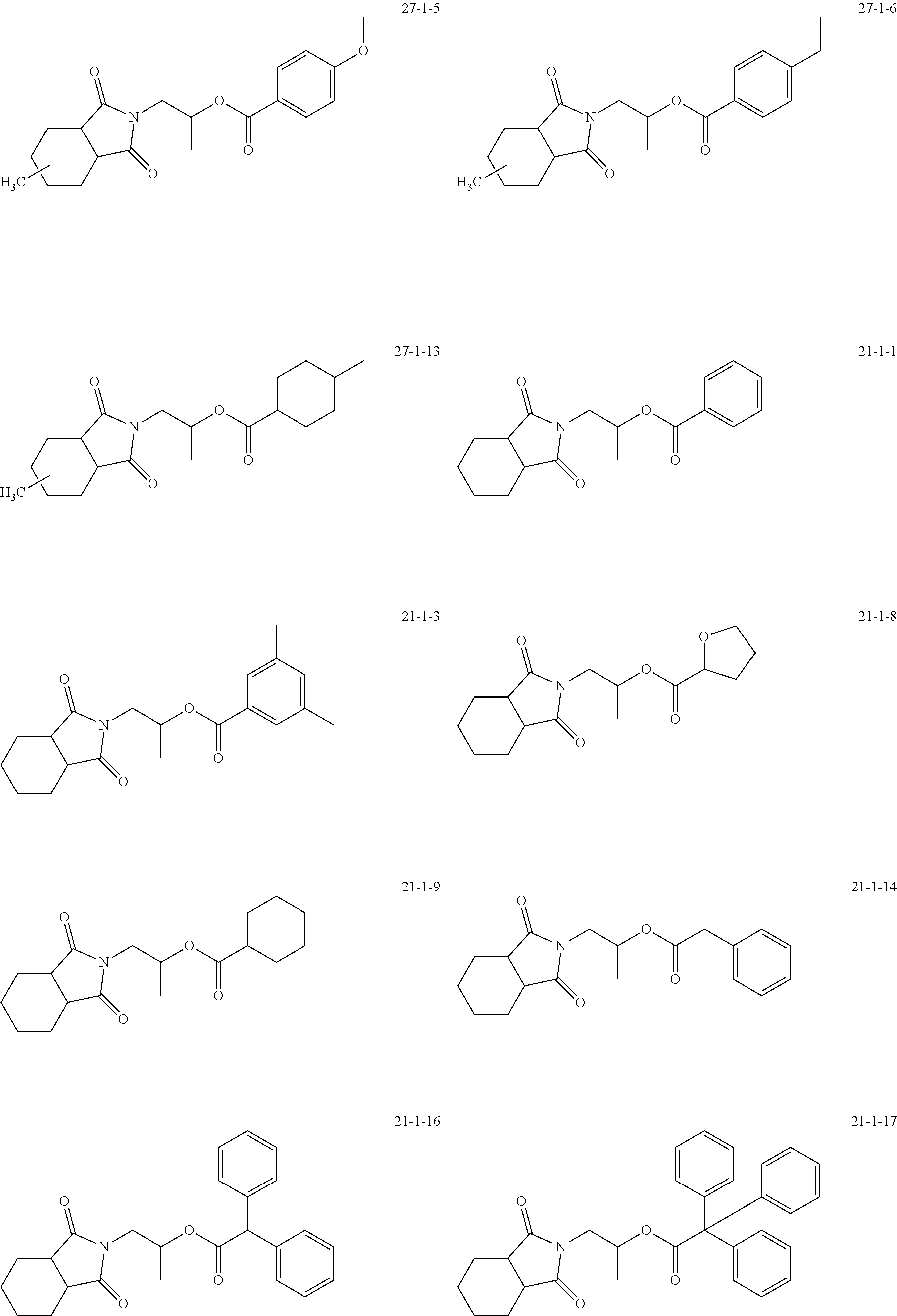

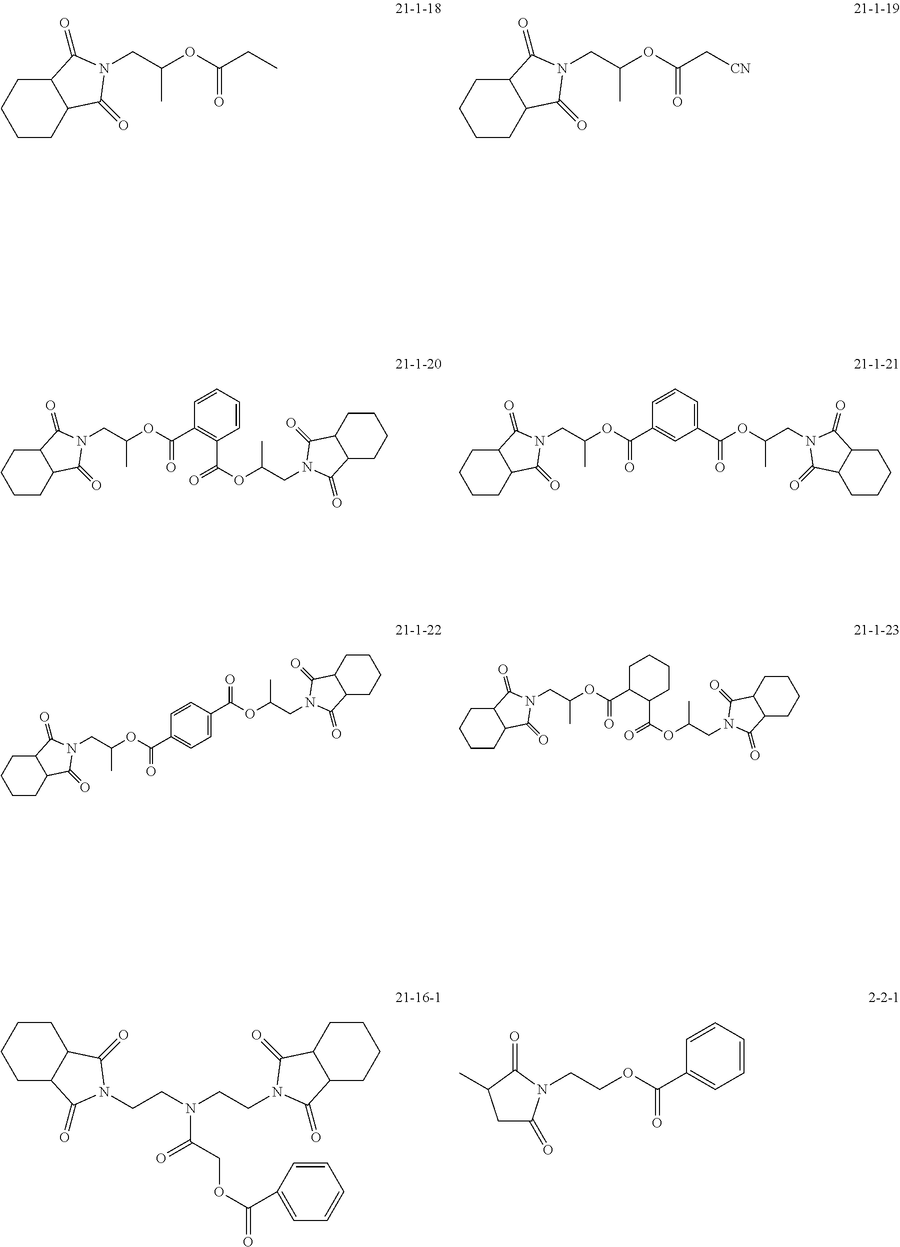

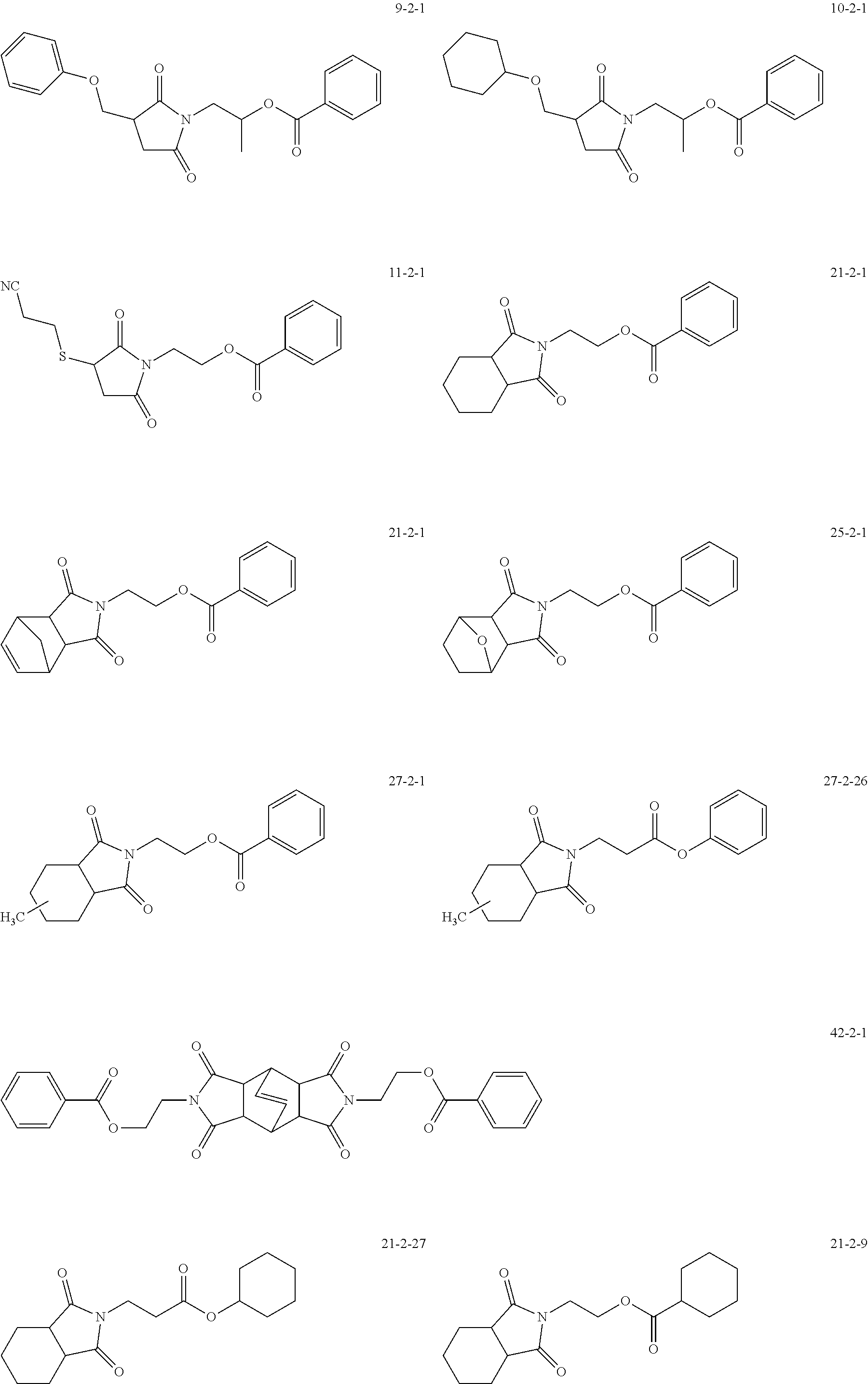

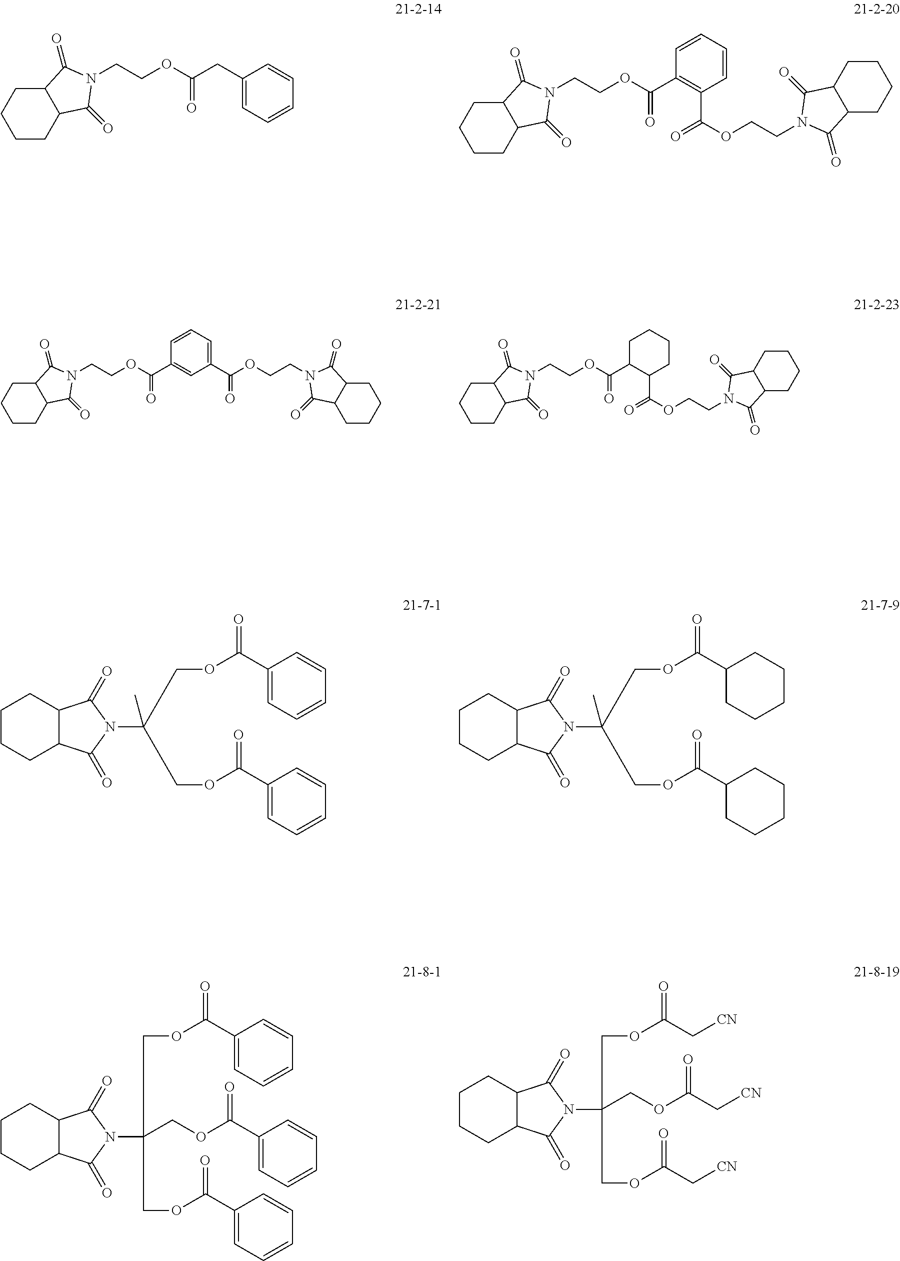

The compound represented by Formula (I) can be produced through a well-known method.

For example, the compound represented by Formula (I-1) can be obtained through a relevant dehydration condensation reaction or the like between acid anhydride and amine. In addition, the compound represented by Formula (I-4) can be obtained by forming a structure represented by Formula (III) through a reaction between alcohol and acid chloride after forming a structure represented by Formula (II) through a relevant dehydration condensation reaction between acid anhydride and amine.

The molecular weight of the compound represented by Formula (I) is preferably 300 to 1000, more preferably 300 to 700, and still more preferably 300 to 600.

The compound having a molecular weight of greater than or equal to 300 is excellent in suppressing vaporization from a film. If the compound has a molecular weight of less than or equal to 1000, compatibility with cellulose acylate becomes sufficient and it is possible to prevent an increase in haze, which is preferable.

The additive amount of the compound represented by Formula (I) is not particularly limited, but is preferably 3 parts by mass to 60 parts by mass, more preferably 5 parts by mass to 40 parts by mass, still more preferably 5 parts by mass to 20 parts by mass, and particularly preferably 5 parts by mass to 15 parts by mass, with respect to 100 parts by mass of cellulose acylate. Two or more kinds of the compounds represented by Formula (I) may be added. In the case of adding two or more kinds thereof, the total amount preferably becomes the above-described additive amount.

(Cellulose Acylate)

As cellulose acylate used in the present invention, ester of cellulose and a fatty acid (including an aromatic fatty acid) is preferable, and cellulose acylate which is acylated by a hydroxyl group being substituted with an acyl group of the above-described fatty acid at 2-position, 3-position, and 6-position of a β-1,4-bonded glucose unit constituting cellulose is preferable. Examples thereof include alkylcarbonyl ester of cellulose, alkenylcarbonyl ester, aromatic carbonyl ester, and aromatic alkyl carbonyl ester. In addition, cellulose acylate in which two or more kinds of acyl groups of fatty acids are substituted is also preferable. These kinds of cellulose acylate may further have a substituted group. As the acyl group with which the hydroxyl group is substituted, it is possible to preferably use an acyl group having 2 to 22 carbon atoms.

The degree of substitution of cellulose acylate means a proportion at which three hydroxyl groups existing in a constituent unit ((β)1,4-glycoside-bonded glucose) of cellulose are acylated. The substitution degree (acylation degree) can be calculated by measuring the amount of a bonded fatty acid per the mass of the constituent unit of cellulose. In the present invention, the degree of substitution of a cellulose body can be calculated by dissolving a cellulose body in a solvent such as deuterium-substituted dimethyl sulfoxide, measuring a ¹³C-NMR spectrum, and obtaining a peak intensity ratio of carbonyl carbon in an acyl group. It is possible to obtain the degree of substitution thereof through ¹³C-NMR measurement after a remaining hydroxyl group of cellulose acylate is substituted with another acyl group which is different from an acyl group included in cellulose acylate itself. The details of the measurement method are disclosed in Tezuka et al. (Carbohydrate. Res., 273 (1995) 83-91).

The degree of substitution of cellulose acylate used in the present invention is preferably 1.5 to 3.0, more preferably 2.00 to 2.97, still more preferably greater than or equal to 2.50 and less than 2.97, and particularly preferably 2.70 to 2.95.

In addition, in cellulose acylate using only acetyl group as an acyl group of cellulose acylate, the degree of substitution thereof is, in terms of a large hardness improvement effect using the compound represented by Formula (I), preferably 2.0 to 3.0, more preferably 2.3 to 3.0, still more preferably 2.60 to 3.0, further more preferably 2.6 to 2.97, and particularly preferably 2.70 to 2.95.

As the acyl group of cellulose acylate which can be used in the present invention, an acetyl group, a propionyl group, and a butyryl group are particularly preferable, and an acetyl group is more particularly preferable.

Mixed fatty acid ester formed of two or more kinds of acyl groups can be preferably used as cellulose acylate in the present invention. Even in this case, an acyl group having 3 to 4 carbon atoms and an acetyl group are preferable as the acyl group. In addition, in a case of using mixed fatty acid ester, the degree of substitution thereof when containing an acetyl group as the acyl group is preferably less than 2.5, and more preferably less than 1.9. In contrast, the degree of substitution thereof when containing an acyl group having 3 to 4 carbon atoms is preferably 0.1 to 1.5, more preferably 0.2 to 1.2, and particularly preferably 0.5 to 1.1.

In the present invention, two kinds of cellulose acylate which have a different substituent and/or the degree of substitution thereof may be used in combination and mixed together, and a film formed of a plurality of layers made of different types of cellulose acylate may be formed through a co-casting method to be described below.

Furthermore, mixed acid ester which is disclosed in paragraphs 0023 to 0038 in JP2008-20896A and has a fatty acid acyl group and a substituted or unsubstituted aromatic acyl group can also be preferably used in the present invention.

Cellulose acylate used in the present invention preferably has a mass average polymerization degree of 250 to 800 and more preferably has a mass average polymerization degree of 300 to 600. In addition, cellulose acylate used in the present invention preferably has a number average molecular weight of 40000 to 230000, more preferably has a number average molecular weight of 60000 to 230000, and most preferably has a number average molecular weight of 75000 to 200000.

Cellulose acylate used in the present invention can be synthesized using acid anhydride or acid chloride as an acylating agent. In a case where the acylating agent is acid anhydride, an organic acid (for example, acetic acid) or methyl chloride is used as a reaction solvent. In addition, it is possible to use a protonic catalyst such as sulfuric acid as a catalyst. In a case where the acylating agent is acid chloride, it is possible to use a basic compound as a catalyst. In most industrially general synthesis methods, cellulose acylate is synthesized by esterifying cellulose using a mixed organic acid component which contains an organic acid (acetic acid, propionic acid, or butyric acid) corresponding to an acetyl group or another acyl group, or acid anhydride (acetic anhydride, protonic anhydride, or butyric anhydride) thereof.

In the method, in many cases, cellulose such as cotton linters or wood pulp is activated by an organic acid such as acetic acid, and then, is esterified using a liquid mixture of the above-described organic acid component in the presence of a sulfuric acid catalyst. The organic acid anhydride component is generally used in an excess amount with respect to the amount of the hydroxyl group existing in cellulose. In the esterification treatment, hydrolysis reaction (depolymerization reaction) of cellulose main chain ((β)1,4-glycoside bonding) proceeds in addition to the esterification reaction. When the hydrolysis reaction of the main chain proceeds, the polymerization degree of cellulose acylate deteriorates and the physical properties of the optical film to be produced deteriorate. For this reason, the reaction conditions such as the reaction temperature are preferably determined in consideration of the polymerization degree or the molecular weight of obtained cellulose acylate.

The optical film of the present invention may contain other additives in addition to cellulose acylate and the compound represented by Formula (I). Examples of the additives include a plasticizer, an organic acid, a pigment, a polymer, a retardation controlling agent, an ultraviolet absorber, an antioxidant, and a matting agent, which are well known. It is possible to refer to these disclosures in paragraphs 0062 to 0097 in JP2012-155287A, and the contents thereof are incorporated in the present specification. The total formulation amount of these is, with respect to the total amount of cellulose acylate, preferably less than or equal to 50 mass % and more preferably less than or equal to 30 mass %.

The method of producing a film of the present invention is not particularly limited, but the film is preferably produced through a melt film formation method or a solution film formation method and more preferably produced through a solution film formation method.

It is possible to produce an optical film of the present invention similarly to the method of producing a cellulose acylate film which is generally performed together with the melt film formation method and the solution film formation method. For example, it is possible to produce the optical film while referring to JP2006-348123A in relation to the melt film formation and to JP2006-241433A in relation to the solution film formation.

Regarding the details of the method of producing the optical film of the present invention through the solution film formation method, it is possible to refer to the disclosures of paragraphs 0079 to 0104 of JP2009-79213A and paragraphs 0093 and 0094 of JP2012-234159A.

Stretching treatment may be performed on the optical film of the present invention in order to increase the surface hardness.

The direction in which a cellulose acylate film is stretched may be any direction. In general, the direction is a cellulose acylate film transportation direction (MD direction) or a direction (TD direction) orthogonal to the transportation direction. It is particularly preferable that the direction is the direction (TD direction) orthogonal to the cellulose acylate film transportation direction in view of a processing process of a polarizing plate, to be continuously performed, using the above-described cellulose acylate film.

The stretching method in the TD direction is disclosed in, for example, JP1987-115035A (JP-S62-115035A), JP1992-152125A (JP-H04-152125A), JP1992-284211A (JP-H04-284211A), JP1992-298310A (JP-H04-298310A), and JP1999-48271A (JP-H11-48271A), and the contents thereof are incorporated in the present specification. In the case of stretching in the MD direction, a cellulose acylate film is stretched if the winding speed of the cellulose acylate film is made to be faster than the peeling speed of the cellulose acylate film by, for example, controlling the speed of a transportation roller of a cellulose acylate film. In the case of stretching in the TD direction, it is also possible to stretch the cellulose acylate film even by gradually expanding the width of a tenter by transporting the cellulose acylate film while maintaining the width of the cellulose acylate film using the tenter. After drying the cellulose acylate film, it is also possible to stretch the cellulose acylate film using a stretching machine (or preferably the cellulose acylate film is uniaxially stretched using a long stretching machine).

In a case of using a cellulose acylate film as a protective film of a polarizer, it is preferable to dispose a transmission axis of the polarizer and an in-plane slow axis of the cellulose acylate film in parallel in order to suppress light leakage when the polarizing plate is obliquely viewed. In general, the transmission axis of the roll film-like polarizer which is continuously produced is parallel to the width direction of the roll film. Therefore, in order to continuously stick the above-described roll film-like polarizer and a protective film, which is formed of a roll film-like cellulose acylate film, to each other, it is necessary for the in-plane slow axis of the roll film-like protective film to be parallel to the width direction of the cellulose acylate film. Accordingly, it is preferable to stretch the film more in the TD direction. In addition, the stretching treatment may be performed in the middle of a film-forming process, or an original fabric which has been winded through film formation may be subjected to stretching treatment.

The stretch ratio in the TD direction is preferably 5% to 100%, more preferably 5% to 80%, and particularly preferably 5% to 40%. Being unstretched means that the stretch ratio is 0%. The stretching treatment may be performed in the middle of the film-forming process, or an original fabric which has been winded through film formation may be subjected to stretching treatment. In the case of the former, stretching may be performed in a state of including a residual solvent amount, and it is possible to preferably stretch the film by a residual solvent amount=(mass of residual volatile component/mass of film after heat treatment)×100 (mass %) of 0.05 mass % to 50 mass %. It is particularly preferable to perform stretching by 5 mass % to 80 mass % in a state in which the residual solvent amount is 0.05 mass % to 5 mass %.

For the purpose of increasing the surface hardness, the heat treatment disclosed in WO2008/114332A may be carried out in the above-described stretching treatment. In the heat treatment, the stretching direction of the cellulose acylate film can be preferably appropriately adjusted to either the cellulose acylate film transportation direction (MD direction) or the direction (TD direction) orthogonal to the transportation direction.

### <Physical Properties of Optical Film>

Hardness:

The optical film of the present invention contains the compound represented by Formula (I) and has high surface hardness. The surface hardness of the optical film in the present invention is measured using Knoop hardness which can be adjusted according to the type or the content of the compound represented by Formula (I).

Modulus of Elasticity

The optical film (particularly, a cellulose acylate film on which other layers are not stacked) of the present invention shows a practically sufficient modulus of elasticity. The range of the modulus of elasticity is not particularly limited, but is, in view of production suitability and handling properties, preferably 1.0 GPa to 7.0 GPa and more preferably 2.0 GPa to 6.0 GPa. The compound represented by Formula (I) in the present invention has an action of increasing the modulus of elasticity by hydrophobizing an optical film by being added to cellulose acylate, and this point is advantageous in the present invention.

Photoelastic Coefficient

The absolute value of a photoelastic coefficient of an optical film is preferably less than or equal to 8.0×10⁻¹² m²/N, more preferably less than or equal to 6×10⁻¹² m²/N, and still more preferably less than or equal to 5×10⁻¹² m²/N. By making the photoelastic coefficient of the optical film small, when the optical film of the present invention is incorporated into a liquid crystal display device as a polarizing plate protective film, it is possible to suppress the generation of irregularities under a high temperature and high humidity. The photoelastic coefficient is measured and calculated through the following method unless otherwise specified.

The lower limit value of the photomodulus of elasticity is not particularly limited, but is practically greater than or equal to 0.1×10⁻¹² m²/N.

The photoelastic coefficient is measured by cutting an optical film into 3.5 cm×12 cm, measuring Re (633) at no load and each load of 250 g, 500 g, 1000 g, and 1500 g using an ellipsometer (M220, JASCO Corporation), and calculating the photoelastic coefficient from an inclination of a straight line of a change in Re with respect to stress.

Moisture Content:

The moisture content of an optical film can be evaluated by measuring the equilibrium moisture content at a constant temperature and humidity. The equilibrium moisture content is calculated by measuring the moisture content of a sample which has reached equilibrium through a Karl Fischer method after allowing the optical film to stand at the above-described temperature and humidity for 24 hours, and by dividing the moisture content (g) by the mass (g) of the sample.

The moisture content of the optical film at a relative humidity of 80% and at 25° C. is preferably less than or equal to 5 mass %, still more preferably less than or equal to 4 mass %, and still more preferably less than 3 mass %. By making the moisture content of an optical film small, when the optical film of the present invention is incorporated into a liquid crystal display device as a polarizing plate protective film, it is possible to suppress the generation of display irregularities of the liquid crystal display device under a high temperature and high humidity. The lower limit value of the moisture content is not particularly limited, but is practically greater than or equal to 0.1 mass %.

Moisture Permeability

The moisture permeability of an optical film can be evaluated by measuring the mass of steam which passes through a sample for 24 hours in the atmosphere of a temperature of 40° C. and a relative humidity of 90% RH based on a moisture permeability test (cup method) of JIS Z 0208 and converting the measured result to the mass of steam which passes through the sample per area of 1 m² for 24 hours.

The moisture permeability of the optical film is preferably 500 g/m²·day to 2000 g/m²·day, more preferably 900 g/m²·day to 1300 g/m²·day, and particularly preferably 1000 g/m²·day to 1200 g/m²·day.

Haze

The haze of an optical film is preferably less than or equal to 1%, more preferably less than or equal to 0.7%, and particularly preferably less than or equal to 0.5%. Making the haze less than or equal to the above-described upper limit value is advantageous in that the transparency of the optical film becomes higher and the optical film becomes easier to be used as an optical film. The haze is measured and calculated through the following method unless otherwise specified. The lower limit value of the haze is not particularly limited, but is practically greater than or equal to 0.001%.

The haze of an optical film of 40 mm×80 mm is measured in accordance with JIS K7136 using a haze meter (HGM-2DP, Suga Test Instruments Co., Ltd.) under the environment of 25° C. and a relative humidity of 60%.

Film Thickness:

Average film thickness of an optical film can be appropriately determined in accordance with the application, and is, for example, 20 μm to 100 μm. The average film thickness of an optical film is preferably 1 μm to 100 μm, more preferably 5 μm to 80 μm, and still more preferably 10 μm to 70 μm. By making the average thickness of the optical film greater than or equal to 10 μm, handling properties are improved when producing a web-like film, which is preferable. In addition, by making the average thickness of the optical film less than or equal to 70 μm, the optical film is easily adapted to a change in humidity and it is easy to maintain optical properties.

In addition, in a case where the optical film has a structure in which three or more layers are stacked, the film thickness of a core layer is preferably 3 μm to 70 μm and more preferably 5 μm to 60 μm. The film thicknesses of a skin layer A and a skin layer B are more preferably 0.5 μm to 20 μm, particularly preferably 0.5 μm to 10 μm, and most preferably 0.5 μM to 3 μm. The core layer refers to a layer positioned at a central portion in a three-layered structure, and the skin layer refers to a layer positioned outside in the three-layered structure.

Width:

The width of an optical film is preferably 700 mm to 3000 mm, more preferably 1000 mm to 2800 mm, and particularly preferably 1300 to 2500 mm.

Retardation:

With the inclusion of the compound represented by Formula (I), it is possible to reduce retardation (in particular, Rth) of an optical film of the present invention.

In the present specification, Re (λ) and Rth (λ) respectively represent in-plane retardation in a wavelength λ and retardation in a thickness direction. Re (λ) is measured by making light of a wavelength of λ nm incident in a film normal direction in KOBRA 21ADH or WR (manufactured by Oji Scientific Instruments). When selecting a measurement wavelength λ nm, it is possible to measure Re (λ) by exchanging a wavelength selection filter manually or changing a measurement value using a program or the like. In a case where the film to be measured is represented by a uniaxial or biaxial refractive index ellipsoid, Rth (λ) is calculated through the following method.

6 points of the Re (λ) in total are measured by making light of a wavelength of λ nm incident from a direction in which an in-plane slow axis (which is determined by KOBRA 21ADH or WR) is inclined by a 10-degree step from a normal direction up to 50° on a single side with respect to a film normal direction in which the in-plane slow axis is set as an inclination axis (rotational axis) (in a case where there is no slow axis, an arbitrary in-plane direction of the film is set to a rotational axis), and Rth (λ) is calculated by KOBRA 21ADH or WR based on the measured retardation value, an assumptive value of an average refractive index, and an input film thickness value. In the above, in a case of a film having a direction in which a retardation value becomes zero at a certain inclination angle by having the in-plane slow axis from the normal direction as a rotational axis, the sign of a retardation value at an inclination angle larger than the inclination angle is changed to minus, and then, KOBRA 21ADH or WR is calculated. The retardation value can be measured from two arbitrary inclined directions by having the slow axis as an inclination axis (rotational axis) (in a case where there is no slow axis, an arbitrary in-plane direction of the film is set to a rotational axis) and Rth can be calculated by the following Formula (A) and Formula (III) based on the measured value, an assumptive value of an average refractive index, and an input film thickness value.

$$Re ( θ ) = [ nx - \frac{( ny \times nz )}{( \sqrt{\begin{matrix}{( ny sin ( {sin}^{- 1} ( \frac{sin ( - θ )}{nx} ) ) )}^{2} + \\ {( nz cos ( {sin}^{- 1} ( \frac{sin ( - θ )}{nx} ) ) )}^{2}\end{matrix}} )} ] \times \frac{d}{cos ( {sin}^{- 1} ( \frac{sin ( - θ )}{nx} ) )} \qquad For mul a ( A )$$

The above-described Re (θ) represents a retardation value in which the slow axis is inclined by an angle θ from the normal direction. In addition, nx in the Formula (A) represents a refractive index in an in-plane slow axis direction, ny represents a refractive index in a direction orthogonal to nx in a plane, and nz represents a refractive index in a direction orthogonal to nx and ny. d represents a film thickness.

Rth={(nx+ny)/2−nz}×d Formula (III)

In a case where the film to be measured is a film which cannot be represented by a uniaxial or biaxial refractive index ellipsoid, that is, a film which does not have an optic axis, Rth (λ) is calculated through the following method. 11 points of the Re (λ) are measured by making light of a wavelength of λ nm incident from a direction in which an in-plane slow axis (which is determined by KOBRA 21ADH or WR) is inclined by a 10° step from −50° to +50° with respect to a film normal direction in which the in-plane slow axis is set as an inclination axis (rotational axis), and Rth (λ) is calculated by KOBRA 21ADH or WR based on the measured retardation value, an assumptive value of an average refractive index, and an input film thickness value. In addition, in the above-described measurement, values of Polymer Handbook (John Wiley & Sons, Inc.) and of catalogs of various optical films can be used as the assumptive values of the average refractive index. It is possible to measure a value of an average refractive index which has not been known, using Abbe's refractometer. Values of the average refractive indexes of primary optical films are exemplified below:

Examples thereof include cellulose acylate (1.48), cycloolefin polymer (1.52), polycarbonate (1.59), polymethyl methacrylate (1.49), and polystyrene (1.59).

By inputting assumptive values of these average refractive indexes and the film thickness, nx, ny, and nz are calculated by KOBRA 21ADH or WR. Nz=(nx −nz)/(nx−ny) is further calculated from these calculated nx, ny, and nz.

The measurement wavelength of the refractive index is set to λ=550 nm in a visible light region unless otherwise specified and the measurement wavelength of Re and Rth is 550 nm unless otherwise specified.

<Saponification Treatment>

It is possible to impart adhesiveness with a material of a polarizer such as polyvinyl alcohol, to the optical film by subjecting the optical film to alkali saponification treatment, and to use the optical film as a polarizing plate protective film.

Regarding the saponification method, it is possible to use a method disclosed in paragraph 0211 and paragraph 0212 in JP2007-86748A.

For example, an alkali saponification treatment with respect to an optical film is preferably performed in a cycle in which the surface of the film is immersed in an alkali solution and is then neutralized with an acid solution, washed with water, and dried. Examples of the alkali solution include a potassium hydroxide solution and a sodium hydroxide solution, and the concentration of hydroxide ions is preferably within a range of 0.1 mol/L to 5.0 mol/L and still more preferably within a range of 0.5 mol/L to 4.0 mol/L. The temperature of the alkali solution is preferably within a range of room temperature to 90° C. and still more preferably within a range of 40° C. to 70° C.

Easy adhesion processing disclosed in JP1994-94915A (JP-H06-94915A) and JP1994-118232A (JP-H06-118232A) may be performed instead of the alkali saponification treatment.

<Polarizing Plate>

Next, a mode of using an optical film of the present invention as a protective film of a polarizing plate will be described. For example, the polarizing plate of the present invention is formed of a polarizer and two sheets of polarizing plate protective film (transparent film) which protect both surfaces of the polarizer, and has the optical film of the present invention as at least one polarizing plate protective film.

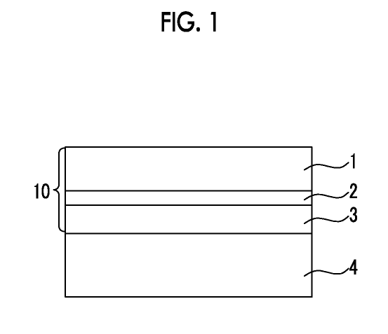

Particularly, the optical film of the present invention is preferably used as a viewer-side protective film of an upper side polarizing plate 10. FIG. 1 is an example showing a positional relationship between a polarizing plate and a liquid crystal display device of the present invention. 1 represents the optical film of the present invention, 2 represents a polarizer, 3 represents a phase difference film, and 4 represents a liquid crystal cell. The phase difference film may be another optical film which is different from the optical film of the present invention. In addition, the upper side of FIG. 1 becomes a viewer-side.

As is exemplified in FIG. 1, the phase difference film 3 is preferably used as a polarizing plate protective film on a side on which the optical film of the present invention is not used. As the phase difference film, a phase difference film, in which a desired phase difference is expressed by formulating various additives with the optical film or stretching the optical film, or a phase difference film, which has an optical anisotropic layer formed of a liquid crystal composition on the surface of a support body, is exemplified. Specifically, it is possible to refer to a disclosure of JP2008-262161A, and the content thereof is incorporated into the present specification.

In addition, as the polarizer, for example, a polarizer obtained by immersing a polyvinyl alcohol film in an iodine solution and stretching the polyvinyl alcohol film is used. In a case of using the polarizer which is obtained by immersing the polyvinyl alcohol film in the iodine solution and stretching the polyvinyl alcohol film, it is possible to directly stick a surface-treated surface of the optical film of the present invention to at least one surface of the polarizer using an adhesive agent. As the adhesive agent, it is possible to use a polyvinyl alcohol aqueous solution or a polyvinyl acetal (for example, polyvinyl butyral) aqueous solution, or latex of a vinyl polymer (for example, polybutyl acrylate). A particularly preferable adhesive agent is a completely saponificated polyvinyl alcohol aqueous solution.

When sticking the optical film of the present invention to the polarizer, it is preferable to stick the optical film thereto such that a transmission axis of the polarizer and a slow axis of the polarizing plate protective film become substantially parallel to each other. It is possible to measure the slow axis through various well-known methods and to perform the measurement of the slow axis using, for example, a birefringence meter (KOBRA 21ADH, manufactured by Oji Scientific Instruments).

Here, being substantially parallel to each other indicates that the deviation between the direction of a main refractive index nx of the polarizing plate protective film and the direction of the transmission axis of the polarizing plate is within 5°, and is preferably within 1° and more preferably within 0.5°. If the deviation therebetween is within 1°, polarization degree performance is hardly reduced under crossed Nicol of the polarizing plate and light leakage hardly occurs, which is preferable.

<Functionalization of Polarizing Plate>

The polarizing plate of the present invention is preferably used as a functionalized polarizing plate which is combined with an anti-reflection film for improving visibility of a display, a film for improving brightness, or an optical film having functional layers such as a hard coat layer, a forward scattering layer, an antiglare (glareproof) layer, and the like, within a range not departing from the gist of the present invention. As the details thereof, it is possible to refer to the disclosures in paragraphs 0229 to 0242 and paragraphs 0249 to 0250 of JP2012-082235A and paragraphs 0086 to 0103 of JP2012-215812A, and the contents thereof are incorporated in the present specification.

<Hard Coat Layer>

The optical film or the polarizing plate of the present invention preferably has a hard coat layer and more preferably has the hard coat layer on the outermost surface of the polarizing plate (viewer-side outermost surface of the liquid crystal display device).

As the hard coat layer, it is possible to refer to the disclosures in paragraphs 0229 to 0242 and paragraphs 0249 to 0250 of JP2012-082235A and paragraphs 0086 to 0103 of JP2012-215812A, and the contents thereof are incorporated in the present specification. The same applies to preferred characteristics, preferred materials, or the like of the hard coat layer which can be used in the present invention.

The thickness of the hard coat layer is preferably 1 μm to 20 μm and more preferably 2 μm to 10 μm.

<Liquid Crystal Display Device>

The liquid crystal display device of the present invention includes at least one sheet of the polarizing plates of the present invention. It is possible to refer to paragraphs 0251 to 0260 of JP2012-082235A for the details of the liquid crystal display device, and the content thereof is incorporated in the present specification.

### EXAMPLES

The present invention will be more specifically described using the following examples. The material, the amount used, the proportion, the processing content, the processing procedure, and the like shown in the following examples can be appropriately modified as long as these do not depart from the gist of the present invention. Accordingly, the range of the present invention is not limited to specific examples shown below.

Identification of all synthesized compounds was performed using ¹H-NMR (300 MHz). In addition, the melting point was measured using a micro melting point measurement apparatus (manufactured by Yanaco New Science Inc., MP-500D).

### Synthesis Example 1

### Synthesis of Compound (21-1)

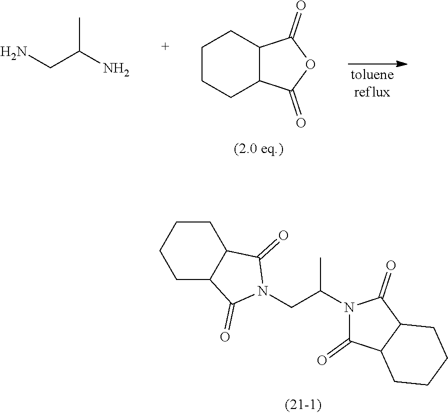

10.0 g (0.135 mol) of cis-cyclohexanedicarboxylic anhydride and 30 mL of toluene were stirred and dissolved in a three neck flask, to which a thermometer, a reflux cooling pipe, and an agitator were attached, at room temperature. The temperature of the mixture was cooled in a bath at 0° C. and 43.7 g (0.283 mol) of 1,2-propanediamine was dripped thereto over 15 minutes. After completion of dripping, the reaction liquid was heated to 130° C. and was stirred for 4 hours. The reaction liquid was concentrated and the concentrate was purified through silica gel column chromatography to obtain a compound (21-1). The yield point was 16 g and the yield coefficient was 34%.

¹H-NMR (CDCl₃) δ1.5-1.2 (m, 10H), 1.9-1.6 (m, 9H), 2.9-2.6 (m, 4H), 3.5 (dd, J=18.4 Hz, 3.2 Hz, 1H), 4.2-4.0 (m, 1H), 4.5-4.3 (m, 1H)

Imide compounds represented by Examples (1-2), (1-4) to (1-13), and (1-21) to (1-22), and Comparative Example 2 were synthesized through the same method as that of Synthesis Example 1.

The imide compounds represented by Examples (1-16) and (1-17) formed imide through the same method as that of Synthesis Example 1 using diamine and itaconic anhydride, and were then synthesized through the method disclosed in Chemistry-An Asian Journal, 4, 1741 (2009).

### Synthesis Example 2

### Synthesis of Compound (21-2-1)

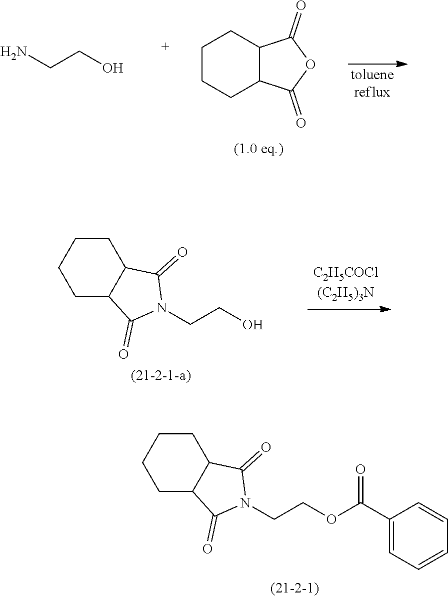

(Step 1)

50.0 g (0.324 mol) of cis-cyclohexanedicarboxylic anhydride and 325 mL of toluene were stirred in a three neck flask, to which a thermometer, a reflux cooling pipe, and an agitator were attached, at room temperature. After the raw materials were dissolved, the temperature of the raw materials was cooled in a bath at 0° C. and 19.8 g (0.324 mol) of 2-aminoethanol was dripped thereto over 15 minutes. Thereafter, the raw materials were heated to 130° C. and were stirred for 4 hours. 200 mL of ethyl acetate was added to the reaction liquid and the mixture was then washed with 1N hydrochloric acid, water, sodium bicarbonate water, and saturated saline solution. The washed mixture was dried over magnesium sulfate and was concentrated to obtain (21-2-1-a). The yield point was 63 g and the yield coefficient was 93%.

(Step 2)

30 g of a compound (21-2-1-a), 300 mL of ethyl acetate, and 16.9 g (0.167 mol) of triethylamine were stirred in a three neck flask, to which a thermometer, a reflux cooling pipe, and an agitator were attached, at room temperature. The temperature of the reaction liquid was cooled to 0° C. 23.5 g (0.167 mol) of benzoyl chloride was dripped thereto, and then the mixture was stirred over 1 hour at room temperature. After the reaction, 200 mL of ethyl acetate was added to the reaction liquid and the mixture was then washed with 1N hydrochloric acid, water, sodium bicarbonate water, and saturated saline solution. The washed mixture was dried over magnesium sulfate and was concentrated. The concentrate was purified through silica gel column chromatography to obtain a compound (21-2-1). The yield point was 35 g and the yield coefficient was 76%.

¹H-NMR (CDCl₃) δ1.5-1.3 (m, 4H), 2.0-1.7 (m, 4H), 3.0-2.9 (m, 2H), 3.9 (t, J=5.4 Hz, 2H), 4.5 (t, J=5.4 Hz, 2H), 7.4 (t, J=7.8 Hz, 2H), 7.6 (t, J=7.2 Hz, 1H), 4.5 (d, J=7.2 Hz, 2H)

Imide compounds represented by Examples (1-14) and (1-15), (1-18) to (1-20), and (1-23) to (1-26) were synthesized through the same method as that of Synthesis Example 2.

### Example 1

### Production of Optical Film

<<<Preparation of Cellulose Acylate Solution>>>

The following composition was put into a mixing tank and was stirred and components were dissolved therein to prepare a cellulose acylate solution.

Composition of cellulose acylate solution

| | | |
|---|---|---|
| Cellulose acylate with acetyl substitution | 100.0 | parts by mass |
| degree of 2.87 and polymerization degree of 370 | | |
| Exemplary compound (21-1) | 10.0 | parts by mass |
| Methylene chloride (first solvent) | 353.9 | parts by mass |
| Methanol (second solvent) | 89.6 | parts by mass |
| n-Butanol (third solvent) | 4.5 | parts by mass |

The prepared cellulose acylate solution was cast using a drum casting apparatus. The cast solution was peeled off in a state where the amount of residual solvent in a dope was about 70 mass % and was dried in a state where the amount of residual solvent was 3 mass % to 5 mass %. Thereafter, the dried solution was further dried by being transported between rolls of a heat treatment apparatus to obtain a cellulose acylate film of Example 1-1. The film thickness of the produced cellulose acylate film was 60 μm. Cellulose acylate films of Examples 1-2 to 1-26 and Comparative Examples 1 to 4 were prepared similarly to Example 1-1 except that the exemplary compound 21-1 was changed to a compound disclosed in Table 1.

<<Preparation of Optical Film with Hard Coat Layer>>

A hard coat layer solution having the following curable composition was applied to the surface of a single-layered optical film formed of each cellulose acylate produced above, the applied solution was irradiated with an ultraviolet ray for curing, and each optical film with a hard coat layer in which the hard coat layer having a thickness of 6 μm was formed was prepared.

Curable composition of hard coat layer solution

| | | |
|---|---|---|
| Monomer pentaerythritol triacrylate/pentaeryth- | 53.5 | parts by mass |
| ritol tetraacrylate (mixed mass ratio 3/2) | | |
| UV initiator Irgacure TM907 (manufactured | 1.5 | parts by mass |
| by BASF Japan Ltd.) | | |
| Ethyl acetate | 45 | parts by mass |

<Evaluation>

<<Evaluation of Retardation Re and Rth>>

The optical film was measured at a wavelength of 550 nm using KOBRA 21ADH.

<<Measurement of Surface Hardness (Knoop Hardness) of Optical Film>>

“Fischer's scope H100Vp type hardness meter” manufactured by Fischer Instruments K.K. was used to measure the surface of a sample fixed to a glass substrate through Knoop indenter of which the direction of a minor axis was disposed parallel to a transportation direction (longitudinal direction: a test direction in a pencil hardness test) during production of a cellulose acylate film, under the conditions of a loading time of 10 sec, a creep time of 5 sec, an unloading time of 10 sec, and a maximum load of 50 mN. Hardnesses were calculated from the relationship between the maximum load and the contact area, which was between the sample and the indenter and obtained from the pushing depth, and an average value of these 5 points was set to surface hardness.

“Fischer's scope H100Vp type hardness meter” manufactured by Fischer Instruments K.K. was used to measure the surface of a sample fixed to a glass substrate based on the method of JIS Z 2251 under the conditions of a loading time of 10 sec, a creep time of 5 sec, an unloading time of 10 sec, and a pushing load of 50 mN. Hardnesses were calculated from the relationship between the maximum load and the contact area, which was between the sample and the indenter and obtained from the pushing depth. JIS Z 2251 is the Japanese Industrial Standards which is made based on ISO 4545.

Furthermore, measurement of omnidirectional Knoop hardness was performed by measuring the Knoop hardness by rotating the Knoop indenter at 18 azimuth equal angles in total which was measured by rotating the Knoop indenter by 10° at the same pushing position, and the minimum value was obtained. As a result, the Knoop hardness was coincident with the surface hardness which was measured by disposing the direction of the minor axis of the above-described Knoop indenter to be parallel to the transportation direction (longitudinal direction: the test direction in a pencil hardness test) during production of a cellulose acylate film. The unit was represented by N/mm², and the results which have been evaluated according to the following criteria are disclosed in the table.

A: Knoop hardness of greater than or equal to 210 N/mm²
B: Knoop hardness of greater than or equal to 205 N/mm² and less than 210 N/mm²
C: Knoop hardness of greater than or equal to 200 N/mm² and less than 205 N/mm²
D: Knoop hardness of greater than or equal to 195 N/mm² and less than 200 N/mm²
E: Knoop hardness of less than 195 N/mm²

<<Method of Evaluating Light-Resistant Adhesiveness>>

A crosscut test in compliance with JIS K 5600 was performed on each above-described optical film with a hard coat layer. Specifically, each optical film with a hard coat layer which had been cured was irradiated with a Xe lamp for 48 hours. 100 lattice patterns with a 1 mm square were made by making 11 cuts longitudinally and 11 cuts horizontally on the hard coat layer after the irradiation with the Xe lamp at 1 mm intervals. Cellophane tape and Mylar tape were stuck thereon and an operation of expeditiously peeling off the cellophane tape and the Mylar tape was repeated three times to evaluate the adhesiveness by visually observing the peeled off site. The irradiation of Xe was performed using super xenon weather meter SX 75 manufactured by Suga Test Instruments Co., Ltd.

The adhesiveness was evaluated based on the following criteria. When the evaluation is superior to or equal to “B”, the adhesiveness between a cellulose acylate film and a hard coat layer is high, and excellent light durability exceeding light durability which is practically required is exhibited.

A: Peeled off site of 0 squares to 5 squares
B: Peeled off site of 6 squares to 15 squares
C: Peeled off site of 16 squares to 25 squares
D: Peeled off site of 26 squares to 35 squares
E: Peeled off site of greater than or equal to 36 squares

<<Haze Evaluation>>

A haze of each optical film obtained from the above was measured and evaluated as A to C.

A: Haze is less than 0.5%
B: Haze is greater than or equal to 0.5% and less than 0.7%
C: Haze is greater than or equal to 0.7%

<<Comprehensive Evaluation>>

When A, B, C, D, and E were respectively set to 5 points, 4 points, 3 points, 2 points, and 1 point in the Knoop hardness evaluation and the light-resistant adhesiveness evaluation and A, B, and C were respectively set to 5 points, 3 points, and 1 point in the haze evaluation, comprehensive evaluation was made from A to E by the total points of the Knoop hardness evaluation point, the light-resistant adhesiveness evaluation point, and the surface evaluation point.

A: Total points are 15 points to 14 points
B: Total points are 13 points to 12 points
C: Total points are 11 points to 9 points
D: Total points are 8 points to 7 points
E: Total points are less than or equal to 6 points

### Other Examples and Comparative Examples

Optical films were obtained in examples and comparative examples under the same conditions except that the compound represented by Formula (I) was changed to others as disclosed in the following table in Example 1.

TABLE 1

| | Compound | | | | | | | |
|---|---|---|---|---|---|---|---|---|
| | represented by | | | Knoop hardness | | | | |
| | Formula (I) or | | | Measured | | Light- | | Compre- |
| | substitute | Re (550) | Rth (550) | value | | resistant | Haze | hensive |
| Example | compound | (nm) | (nm) | (N/mm²) | Evaluation | adhesiveness | evaluation | evaluation |
| Example 1-1 | 21-1 | 2 | −10 | 212 | A | A | A | A |
| Example 1-2 | 21-9 | 1 | −19 | 211 | A | A | A | A |
| Example 1-3 | 21-2-1 | 0 | 12 | 200 | C | A | A | B |
| Example 1-4 | 23-2 | 2 | −16 | 208 | B | A | A | A |
| Example 1-5 | 21-12 | 1 | −9 | 215 | A | A | A | A |
| Example 1-6 | 21-15 | 0 | −3 | 209 | B | A | A | A |
| Example 1-7 | 1-12 | 2 | 2 | 202 | C | A | A | B |
| Example 1-8 | 24-1 | 1 | −18 | 211 | A | A | A | A |
| Example 1-9 | 26-13 | 0 | 9 | 214 | A | A | A | A |
| Example 1-10 | 21-13 | 1 | −7 | 212 | A | A | A | A |
| Example 1-11 | 21-3 | 2 | −8 | 201 | C | A | A | B |
| Example 1-12 | 21-10 | 1 | −17 | 216 | A | A | A | A |
| Example 1-13 | 21-14 | 0 | 23 | 218 | A | B | A | A |
| Example 1-14 | 21-8-1 | 0 | 73 | 215 | A | A | A | A |
| Example 1-15 | 21-2-20 | 0 | 0 | 212 | A | B | A | A |
| Example 1-16 | 15-10 | 0 | 20 | 204 | C | A | A | B |
| Example 1-17 | 16-13 | 0 | 10 | 201 | C | A | A | B |
| Example 1-18 | 42-2-1 | 1 | 26 | 198 | D | B | B | C |
| Example 1-19 | 21-7-1 | 1 | 25 | 212 | A | A | A | A |
| Example 1-20 | 21-8-19 | 1 | 15 | 225 | A | A | A | A |
| Example 1-21 | 21-2-27 | 1 | −2 | 201 | C | A | A | B |
| Example 1-22 | 28-1 | 1 | −5 | 213 | A | A | A | A |
| Example 1-23 | 27-1-6 | 0 | 1 | 210 | A | A | A | A |
| Example 1-24 | 27-1-5 | 0 | 5 | 211 | A | B | A | A |
| Example 1-25 | 21-1-9 | 0 | −8 | 211 | A | A | A | A |
| Example 1-26 | 27-1-13 | 0 | −8 | 211 | A | A | A | A |
| Comparative | Comparative | 0 | 11 | 199 | D | D | C | E |
| Example 1 | compound 1 | | | | | | | |
| Comparative | Comparative | 0 | 43 | 195 | D | E | B | D |
| Example 2 | compound 2 | | | | | | | |
| Comparative | Comparative | — | — | 203 | C | E | C | E |
| Example 3 | compound 3 | | | | | | | |
| Comparative | None | 0.3 | 25 | 180 | E | A | B | C |
| Example 4 | | | | | | | | |

Comparative Compound 1 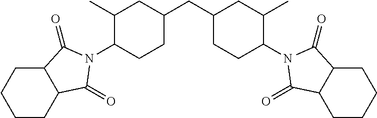 [SMILES: CC1CC(CC2CCC(N3C(=O)C4CCCCC4C3=O)C(C)C2)CCC1N1C(=O)C2CCCCC2C1=O]
Comparative Compound 2 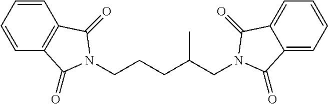 [SMILES: CC(CCCN1C(=O)c2ccccc2C1=O)CN1C(=O)c2ccccc2C1=O]
Comparative Compound 3

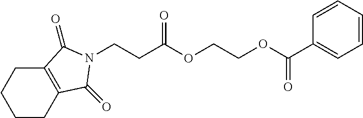 [SMILES: O=C(CCN1C(=O)C2=C(CCCC2)C1=O)OCCOC(=O)c1ccccc1]

Comparative compound 1 is A-089 disclosed in JP2012-123292A. Comparative Compound 2 was synthesized through a method of Synthesis Example 1. Comparative Compound 3 is Compound 4-5 disclosed in JP2009-15045A.

As is obvious from the above-described table, it was found that more favorable light-resistant adhesiveness was exhibited compared to the case in which Comparative Compounds 1 to 3 were added, by producing a film by formulating the compound represented by Formula (I) with cellulose acylate. In addition, it was found that the Knoop hardness was more improved than that in Comparative Example 4 and the compound represented by Formula (I) had an effect of improving the surface hardness of the film. Furthermore, it was found that the surface of the film was better (haze was low) than those in Comparative Examples 1 and 3. In Comparative Example 1, it is considered that there are many saturated fat rings in an additive and the compatibility with cellulose acylate is inferior. In Comparative Examples 2 and 3, there is an unsaturated bond in an imide ring. Therefore, it is considered that the light-resistant adhesiveness deteriorates as a result of cellulose acylate decomposed in the light resistance test.

Regarding Re and Rth in Comparative Example 3, it was impossible to measure Re and Rth due to the inferior surface of the film.

(Evaluation of Pencil Hardness)

The moisture of each cellulose acylate film with a hard coat layer was adjusted for 2 hours under the conditions of 25° C. and relative humidity of 60%. Then, the surface of the hard coat layer was repeatedly scratched by a pencil at each hardness 5 times with a weight of 500 g using a test pencil specified by JIS-S 6006 in accordance with a pencil hardness evaluation method specified by JIS-K 5400, and the hardness was measured until one scratch was generated. There is a disclosure that the scratch defined by JIS-K 5400 is a tear or a scratch on a coated film, and a dent of a coated film is not regarded as an object. However, in this evaluation, it was determined that the dent of a coated film is included in the scratch. Practically, greater than or equal to 3H is preferable, and the higher the numerical value is, the higher the hardness is, which is preferable. Pencil hardness evaluation was carried out using cellulose acylate films of Examples 1-1, 1-2, and 1-10. As a result, it was found that all of the cellulose acylate films graded 3H which was being highly evaluated.

### Example 2

Cellulose acylate films of Examples 2-1 to 2-7 were produced similarly to Example 1 except that the degree of substitution of cellulose acylate and the kinds of additives were changed to others as shown in the following table.

The evaluation of the characteristics was performed similarly to that in Example 1.

TABLE 2

| | Cellulose | Compound represented by | | | | | |
|---|---|---|---|---|---|---|---|
| | acylate | Formula (I) | | | | | |
| | Degree of | | Additive | Knoop | Light- | | Compre- |
| | substitution | Compound | amount (parts | hardness | resistant | Surface | hensive |
| | of acetyl | number | by mass) | evaluation | adhesiveness | evaluation | evaluation |
| Example 2-1 | 2.42 | 21-1 | 10 | A | A | A | A |
| Example 2-2 | 2.42 | 21-9 | 10 | A | A | A | A |
| Example 2-3 | 2.42 | 27-1-6 | 10 | A | A | A | A |
| Example 2-4 | 2.77 | 21-12 | 10 | A | A | A | A |
| Example 2-5 | 2.93 | 21-1 | 10 | A | A | A | A |
| Example 2-6 | 2.93 | 27-1-6 | 10 | A | A | A | A |
| Example 2-7 | 2.93 | 21-10 | 10 | A | A | A | A |

* The unit of the additive amount of the compound represented by Formula (I) is parts by mass with respect to 100 parts by mass of cellulose acylate.

As disclosed in the above-described table, it was found that preferable Knoop hardness, preferable light-resistant adhesiveness, and a preferable effect of suppressing a haze could be exhibited regardless of the degree of substitution of cellulose acylate in the compound of the present invention.

### Example 3

### Production of Polarizing Plate

Saponification Treatment of Polarizing Plate Protective Film

Cellulose acylate films of Examples 1-1, 1-2, and 1-10 which were obtained in Example 1 were immersed in a 2.3 mol/L sodium hydroxide aqueous solution for 3 minutes at 55° C. The films were washed in a water washing tub at room temperature and were neutralized with 0.05 mol/L sulfuric acid at 30° C. Again, the films were washed in the water washing tub at room temperature and were dried with warm air at 100° C. In this manner, the saponification treatment was performed on the surface of each of the cellulose acylate films. In addition, the same treatment was performed on the produced cellulose acylate films without adding the compound represented by Formula (I) as a comparative example.

Production of Polarizing Plate

Iodine was made to be adsorbed to a stretched polyvinyl alcohol film to produce a polarizer.

The cellulose acylate films which were subjected to the saponification treatment as described above were stuck to a single side of the polarizer using a polyvinyl alcohol-based adhesive agent. The same saponification treatment was performed on commercially available cellulose triacetate films (FUJITAC TD80UF, manufactured by Fujifilm Corporation). The cellulose triacetate films after the saponification treatment were stuck to the surface of the polarizer on a side opposite to the side to which the cellulose acylate films produced above were stuck, using the polyvinyl alcohol-based adhesive agent.

At this time, a transmission axis of the polarizer and a slow axis of an obtained cellulose acylate film were disposed so as to be parallel to each other. In addition, a transmission axis of the polarizer and a slow axis of a commercially available cellulose triacetate film were disposed so as to be orthogonal to each other.

In this manner, a polarizing plate was produced using each of the cellulose acylate films of Examples 1-1, 1-2, and 1-10.

### Example 300

### Production of Liquid Crystal Display Device

A viewer-side polarizing plate of a commercially available liquid crystal television (Bravia J5000 of Sony Corporation) was peeled off. A liquid crystal display device was obtained by sticking each polarizing plate produced in the above-described examples to an observer side through an adhesive agent such that the polarizing plate protective films of Examples 1-1, 1-2, and 1-10 in the above-described examples become a side opposite to a liquid crystal cell side, as the polarizing plate of the present invention. It was confirmed that all of the liquid crystal display devices exhibited favorable display performance.

### EXPLANATION OF REFERENCES

1 optical film of the present invention
2 polarizer
3 phase difference film
4 liquid crystal cell
10 upper side polarizing plate

What is claimed is:

1. An optical film comprising:
cellulose acylate; and
a compound represented by Formula (I);
(A¹)ₙ₁-L-(A²)ₙ₂ Formula (I)
wherein, in Formula (I), L represents a linking group of (n1+n2) valence of which the number of atoms linking A¹ to A² is less than or equal to 8; here, n1+n2 represents an integer of greater than or equal to 2, n1 represents an integer of greater than or equal to 1, and n2 represents an integer of greater than or equal to 0; and A¹ represents a group represented by Formula (II) and A² represents a group represented by Formula (III); 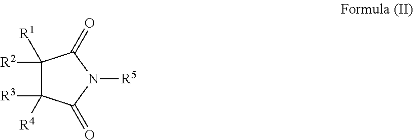
wherein, in Formula (II), R¹ to R⁴ each independently represent a hydrogen atom, a substituted or unsubstituted alkyl group, a substituted or unsubstituted acyl group, a substituted or unsubstituted alkoxycarbonyl group, a substituted or unsubstituted alkoxy group, a substituted or unsubstituted aryloxy group, a substituted or unsubstituted alkylthio group, a substituted or unsubstituted arylthio group, a substituted or unsubstituted carbamoyl group, a carboxyl group, a cyano group, or a hydroxyl group, and R¹ to R⁴ may form a non-aromatic ring by being bonded to each other; and R⁵ represents a hydrogen atom or a substituted or unsubstituted alkyl group; here, one of R¹ to R⁵ represents a bonding site with L in Formula (I); and 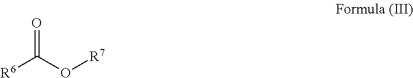
wherein, in Formula (III), R⁶ and R⁷ each independently represent a substituted or unsubstituted phenyl group or a substituted or unsubstituted alkyl group; here, at least one of R⁶ and R⁷ represents a bonding site with L in Formula (I).

2. The optical film according to claim 1,
wherein, in Formula (I), L contains at least one of a tertiary carbon atom and a quaternary carbon atom.

3. The optical film according to claim 1,
wherein, in Formula (I), L represents a linking group of (n1+n2) valence of which the number of atoms linking A¹ to A² is less than or equal to 5.

4. The optical film according to claim 1,
wherein the molecular weight of the compound represented by Formula (I) is 300 to 1000.

5. The optical film according to claim 1,
wherein, in Formula (II), the bonding site with L is R⁵.

6. The optical film according to claim 1,
wherein, in Formula (I), n1 represents an integer of 2 to 5 and n2 represents 0.

7. The optical film according to claim 6,
wherein, in Formula (I), n1 represents an integer of 2 or 3.

8. The optical film according to claim 1,
wherein, in Formula (II), R¹ to R⁴ bond to each other to form a 5- or 6-membered aliphatic ring.

9. The optical film according to claim 1,
wherein, in Formula (I), n1 represents 1 and n2 represents an integer of 1 to 5.

10. The optical film according to claim 1,
wherein, in Formula (III), the bonding site with L is R⁷.

11. The optical film according to claim 1,
wherein, in Formula (II), R¹ to R⁴ each independently represent a hydrogen atom, an alkyl group, an acyl group, an alkoxycarbonyl group, a carbamoyl group, an alkoxy group, an aryloxy group, an alkylthio group, an arylthio group, a cyano group, or a hydroxyl group.

12. The optical film according to claim 1,
wherein, in Formula (III), R⁶ represents a substituted or unsubstituted phenyl group or a substituted or unsubstituted alkyl group.

13. The optical film according to claim 1,
wherein the content of the compound represented by Formula (I) is 3 parts by mass to 60 parts by mass with respect to 100 parts by mass of cellulose acylate.

14. The optical film according to claim 1,
wherein the optical film is used for a protective film of a viewer-side polarizing plate.

15. The optical film according to claim 1, further comprising:
a hard coat layer.

16. A polarizing plate comprising:
the optical film according to claim 1; and
a polarizer.

17. A liquid crystal display device comprising:
the polarizing plate according to claim 16.

18. A compound which is represented by Formula (I-3) or (I-5). 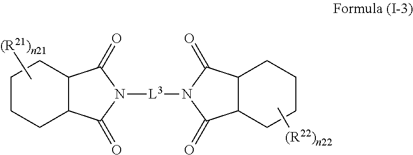
wherein, in Formula (I-3), L³ represents a divalent linking group which contains at least one of a tertiary carbon atom and a quaternary carbon atom and in which the number of atoms linking nitrogen atoms together is less than or equal to 8; R²¹ and R²² represent a methyl group; and n21 and n22 each represent an integer of 0 or 1; 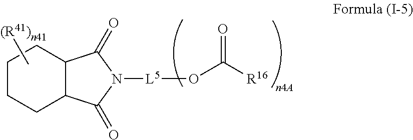
wherein in Formula (I-5), L⁵ represents a linking group which is selected from the following linking group A, R⁴¹ represents a methyl group, and n41 represents an integer of 0 or 1; R¹⁶ represents a substituent which is selected from the following substituent group B and a plurality of the substituents may be the same as or different from each other; and n4A represents an integer of 1 to 3;
Linking group A: 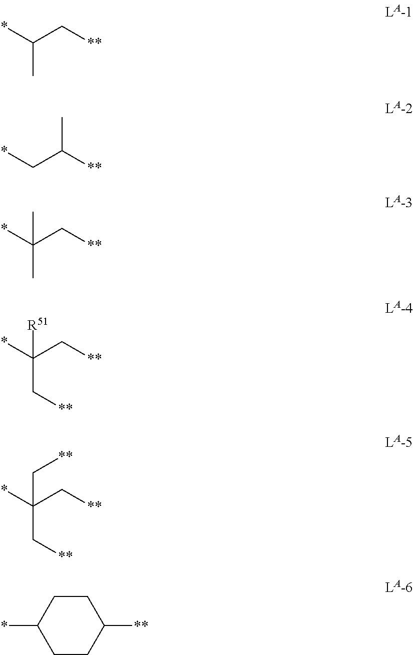
wherein * represents a bonding site with a nitrogen atom and ** represents a bonding site with an oxygen atom; and R⁵¹ represents a hydrogen atom, a methyl group, or an ethyl group; and
Substituent group B: 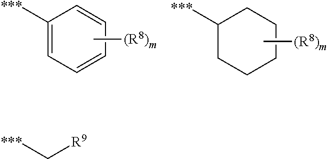
wherein *** represents a bonding site with a carbonyl group, R⁸ represents an alkyl group, an alkoxycarbonyl group, an acyl group, or an alkoxy group, and R⁹ represents a carbonyl group, an alkoxycarbonyl group, a carbonyloxy group, a cyano group, or a hydroxyl group; and m represents an integer of 0 to 3.
